(19) United States
(12) Patent Application Publication
(10) Pub. No.: US 2026/0022185 A1
(43) Pub. Date: Jan. 22, 2026

(54) METHOD OF REDUCING AGGREGATION IN THE VIRUS-INACTIVATED PREPARATION OF ANTI-CD40 ANTIBODIES

(71) Applicant: Kiniksa Pharmaceuticals, GmbH, Zug (CH)

(72) Inventors: Doug Gunzelmann, Lexington, MA (US); Shaun Grier, Lexington, MA (US)

(73) Assignee: Kiniksa Pharmaceuticals, GmbH, Zug (CH)

(21) Appl. No.: 19/008,069

(22) Filed: Jan. 2, 2025

Related U.S. Application Data
(60) Provisional application No. 63/617,366, filed on Jan. 3, 2024.

(51) Int. Cl. C07K 16/28; C12N 7/00

(52) U.S. Cl. CPC C07K 16/2878; C12N 7/00; C07K 2317/24; C12N 2740/13061

(57) ABSTRACT

The instant invention relates to compositions of, and methods for producing, a virus-inactivated antibody preparation having a reduced level of high molecular weight aggregates, and/or methods for minimizing formation of high molecular weight aggregates in a virus-inactivated antibody preparation, comprising an antibody, or antigen-binding portion thereof, e.g., an anti-CD40 antibody or antigen binding portion thereof.

### RELATED APPLICATION

This application claims the benefit of priority to U.S. Provisional Application No. 63/617,366, filed on Jan. 3, 2024, the entire contents of which are incorporated hereby by reference.

### SEQUENCE LISTING

The instant application contains a Sequence Listing which has been submitted electronically in XML format and is hereby incorporated by reference in its entirety. Said XML copy, created Dec. 27, 2024, is named 132261_00202.xml and is 17,440 bytes in size. The sequence listing contained in this .XML file is part of the specification and is hereby incorporated by reference herein in its entirety.

### BACKGROUND OF THE INVENTION

Antibodies, such as monoclonal antibodies, are an important class of therapeutic drugs in the pharmaceutical industry. Antibody therapeutics have been developed for treating many diseases, such as autoinflammatory and autoimmune disorders.

The production of proteins, such as monoclonal antibodies, for pharmaceutical applications generally involves the use of upstream process technologies (e.g., cell culture) and downstream process technologies (e.g., protein purification). Typically, antibodies are produced as recombinant proteins in mammalian cell cultures to ensure proper folding and post-translational modification. Monoclonal antibodies produced from cell cultures need to be purified from host cell impurities, product related impurities, and other process-related impurities in order to be effectively utilized, for example, to improve the safety profile and potency.

The purification process of monoclonal antibodies typically consists of primary recovery, capture (e.g., Protein A chromatography), viral inactivation via low pH or detergent, polishing (e.g., ion exchange chromatography), viral filtration, and ultrafiltration and diafiltration steps.

The viral inactivation step is dedicated exclusively to viral reduction to achieve an effective viral clearance strategy. Low-pH viral inactivation often follows the Protein A capture step. The pool is titrated to a pH sufficiently acidic to disrupt viral envelopes and subjected to a static hold, after which it is titrated to a pH at which the protein is more stable and which is more suitable for the next chromatography step.

The inventors have discovered that the low-pH viral inactivation step unexpectedly induces protein aggregation of anti-CD40 monolclonal antibody during the process. Establishing a robust manufacturing process that minimizes protein aggregation is of paramount importance in the development of therapeutic antibodies. It is necessary in the course of clinical development to establish conditions for viral inactivation without having a negative impact on product quality.

### SUMMARY OF THE INVENTION

The present invention is based, at least, on the identification of surprising and unexpected virus inactivation and neutralization conditions during the purification process of an anti-CD40 antibody, or antigen-binding portions thereof, e.g., KPL-404, that stabilizes the protein and/or reduces formation of high molecular weight aggregates in the virus-inactivated protein preparation.

Typical viral inactivation process used in the industry involves titration of a sample containing a protein of interest to a low pH in order to deactivate any enveloped viruses and viral components. The pH conditions are selected as a balance between a low pH value that is sufficient to cause virus inactivation and a high enough value to avoid denaturation of the protein.

The inventors of the present invention have surprisingly and unexpectedly discovered that despite pH ≤3.6 being the industry standard for the viral inactivation process, a substantial and unacceptable level of protein aggregates was observed during the viral inactivation step for an anti-CD40 antibody, e.g., KPL-404, at this pH range. Accordingly, a new surprising condition for viral inactivation was established during the purification process to ensure sufficient viral inactivation while minimizing proten aggregation.

Accordingly, in one aspect, the present invention provides a method of producing a virus-inactivated antibody preparation comprising an anti-CD40 antibody, or antigen-binding portion thereof, having a reduced level of high molecular weight aggregates, the method comprising incubating a sample comprising the anti-CD40 antibody, or antigen-binding portion thereof, at a pH of about 3.6-3.9 during a viral inactivation step, thereby producing the virus-inactivated antibody preparation having a reduced level of high molecular weight aggregates.

In another aspect, the present invention provides a method of minimizing formation of high molecular weight aggregates in an antibody preparation comprising an anti-CD40 antibody, or antigen-binding portion thereof, during a viral inactivation step, the method comprising incubating a sample comprising the anti-CD40 antibody, or antigen-binding portion thereof, at a pH of about 3.6-3.9 during the viral inactivation step, thereby minimizing formation of high molecular weight aggregates in the virus-inactivated antibody preparation.

In one aspect, the present invention provides a method of reducing formation of high molecular weight aggregates in a antibody preparation comprising an anti-CD40 antibody, or antigen-binding portion thereof, during a viral inactivation step, the method comprising incubating a sample comprising the anti-CD40 antibody, or antigen-binding portion thereof, at a pH of about 3.6-3.9 during the viral inactivation step, thereby reducing the formation of high molecular weight aggregates in the virus-inactivated antibody preparation.

In another aspect, the present invention provides a method of maximizing the level of antibody monomers in a virus-inactivated antibody preparation comprising an anti-CD40 antibody, or antigen-binding portion thereof, the method comprising incubating a sample comprising the anti-CD40 antibody, or antigen-binding portion thereof, at a pH of about 3.6-3.9 during a viral inactivation step, thereby maximizing antibody monomers in the virus-inactivated antibody preparation.

In one aspect, the present invention provides a method of stabilizing a virus-inactivated antibody preparation comprising an anti-CD40 antibody, or antigen-binding portion thereof, the method comprising incubating a sample comprising the anti-CD40 antibody, or antigen-binding portion thereof, at a pH of about 3.6-3.9 during a viral inactivation step, thereby stabilizing the virus-inactivated antibody preparation.

In another aspect, the present invention provides a method of reducing the number and/or activity of viral particles in an antibody preparation comprising an anti-CD40 antibody, or antigen-binding portion thereof, the method comprising incubating a sample comprising the anti-CD40 antibody, or antigen-binding portion thereof, at a pH of about 3.6-3.9 during a viral inactivation step, thereby reducing the level the number and/or activity of viral particles in the antibody preparation.

In some embodiments, the viral inactivation step comprises a virus inactivation period, and optionally a static holding period.

In some embodiments, the anti-CD40 antibody, or antigen binding portion thereof, comprises a heavy chain variable region comprising a CDR1 having an amino acid sequence of SEQ ID NO:1, a CDR2 having an amino acid sequence of SEQ ID NO:2, and a CDR3 having an amino acid sequence of SEQ ID NO:3.

In some embodiments, the anti-CD40 antibody, or antigen binding portion thereof, comprises a light chain variable region comprising a CDR1 having an amino acid sequence of SEQ ID NO:4, a CDR2 having an amino acid sequence of SEQ ID NO:5, and a CDR3 having an amino acid sequence of SEQ ID NO:6.

In some embodiments, the anti-CD40 antibody, or antigen binding portion thereof, comprises a heavy chain variable region comprising an amino acid sequence of SEQ ID NO:7, and a light chain variable region comprising an amino acid sequence of SEQ ID NO:8.

In some embodiments, the anti-CD40 antibody, or antigen binding portion thereof, comprises a heavy chain comprising an amino acid sequence of SEQ ID NO:9, and a light chain comprising an amino acid sequence of SEQ ID NO:10.

In some embodiments, the anti-CD40 antibody or antigen-binding portion thereof is KPL-404.

In some embodiments, the sample is incubated during the viral inactivation step at a pH of about 3.6-3.9, about 3.6-3.8, about 3.7-3.9, about 3.6-3.7, about 3.7-3.8, or about 3.8-3.9.

In some embodiments, the sample is incubated during the viral inactivation step at a pH of about 3.6, about 3.7, about 3.8, or about 3.9.

In some embodiments, the sample is incubated during the viral inactivation step for about 15-360 minutes.

In some embodiments, the sample is incubated during the viral inactivation step for about 15-50 minutes, about 20-40 minutes, about 20-60 minutes, about 30-70 minutes, about 30-60 minutes, about 30-120 minutes, about 60-120 minutes, about 40-80 minutes, about 50-70 minutes, about 50-90 minutes, about 60-100 minutes, about 70-110 minutes, about 80-100 minutes, about 80-120 minutes, about 90-130 minutes, about 100-140 minutes, about 110-130 minutes, about 110-150 minutes, about 120-160 minutes, about 130-170 minutes, about 140-180 minutes, about 150-190 minutes, about 160-200 minutes, about 170-190 minutes, about 170-210 minutes, about 180-220 minutes, about 200-240 minutes, about 220-260 minutes, about 230-250 minutes, about 240-280 minutes, about 280-320 minutes, about 290-310 minutes, or about 320-360 minutes.

In some embodiments, the sample is incubated during the viral inactivation step at a temperature of about 13° C.-37° C., about 15° C.-37° C., about 15° C.-30° C., about 13° C.-25° C., or about 15° C.-25° C.

In some embodiments, the sample is incubated during the viral inactivation step at a pH of about 3.7-3.9 for about 30-120 minutes. In some embodiments, the sample is incubated during the viral inactivation step at a pH of about 3.7-3.9 for about 60-120 minutes. In other embodiments, the sample is incubated during the viral inactivation step at a pH of about 3.7-3.9 at a temperature of about 15° C.-25° C. for about 50-70 minutes.

In some embodiments, the sample is incubated during the viral inactivation step at a pH of about 3.6-3.8 for about 30-120 minutes. In some embodiments, the sample is incubated during the viral inactivation step at a pH of about 3.6-3.8 for about 60-120 minutes. In other embodiments, the sample is incubated during the viral inactivation step at a pH of about 3.6-3.8 at a temperature of about 15° C.-25° C. for about 50-70 minutes.

In some embodiments, the sample is incubated during the viral inactivation step at a pH of about 3.7-3.9 at a temperature of about 15° C.-25° C. for about 30-120 minutes. In some embodiments, the sample is incubated during the viral inactivation step at a pH of about 3.7-3.9 at a temperature of about 15° C.-25° C. for about 60-120 minutes.

In some embodiments, the sample is incubated during the viral inactivation step at a pH of about 3.6-3.8 at a temperature of about 15° C.-25° C. for about 30-120 minutes. In some embodiments, the sample is incubated during the viral inactivation step at a pH of about 3.6-3.8 at a temperature of about 15° C.-25° C. for about 60-120 minutes.

In some embodiments, the sample is incubated during the viral inactivation step at a pH of about 3.7-3.8 at a temperature of about 13° C.-25° C. for about 30-120 minutes.

In some embodiments, the sample is incubated during the viral inactivation step at a pH of about 3.7 for about 60-120 minutes. In some embodiments, the sample incubated during the viral inactivation step at a pH of about 3.7 for about 120 minutes.

In some embodiments, the sample is incubated during the viral inactivation step at a pH of about 3.7 at a temperature of about 15° C.-25° C. for about 60-120 minutes. In some embodiments, the sample is incubated during the viral inactivation step at a pH of about 3.7 at a temperature of about 15° C.-25° C. for about 120 minutes. In some embodiments, the sample is incubated during the viral inactivation step at a pH of about 3.7 at a temperature of about 25° C. for about 120 minutes.

In some embodiments, the sample is incubated during the viral inactivation step at a pH of about 3.8 for about 60-120 minutes. In some embodiments, the sample is incubated during the viral inactivation step at a pH of about 3.8 for about 60 minutes. In other embodiments, the sample is incubated during the viral inactivation step at a pH of about 3.8 at a temperature of about 15° C.-25° C. for about 60 minutes.

In some embodiments, the sample is subjected to a Protein A chromatography column prior to the viral inactivation step.

In some embodiments, the virus-inactivated antibody preparation comprises less than 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% high molecular weight aggregates.

In some embodiments, the virus-inactivated antibody preparation comprises less than 2% high molecular weight aggregates. In some embodiments, the virus-inactivated antibody preparation comprises less than 1% high molecular weight aggregates.

In some embodiments, the level of high molecular weight aggregates is determined by size exclusion chromatography.

In one aspect, the present invention provides a composition comprising an anti-CD40 antibody, or antigen-binding portion thereof, wherein the composition comprises a virus-inactivated eluate collected from an affinity chromatography column, wherein the eluate comprises less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% high molecular weight aggregates.

In some embodiments, the virus-inactivated eluate comprises less than 2% high molecular weight aggregates. In some embodiments, the virus-inactivated eluate comprises less than 1% high molecular weight aggregates.

In some embodiments, the affinity chromatography column comprises a Protein A chromatography column.

In some embodiments, the virus-inactivated eluate has been subjected to a viral inactivation step at a pH of about 3.6-3.9.

In some embodiments, the anti-CD40 antibody, or antigen binding portion thereof, comprises a heavy chain variable region comprising a CDR1 having an amino acid sequence of SEQ ID NO:1, a CDR2 having an amino acid sequence of SEQ ID NO:2, and a CDR3 having an amino acid sequence of SEQ ID NO:3.

In some embodiments, the anti-CD40 antibody, or antigen binding portion thereof, comprises a light chain variable region comprising a CDR1 having an amino acid sequence of SEQ ID NO:4, a CDR2 having an amino acid sequence of SEQ ID NO:5, and a CDR3 having an amino acid sequence of SEQ ID NO:6.

In some embodiments, the anti-CD40 antibody, or antigen binding portion thereof, comprises a heavy chain variable region comprising an amino acid sequence of SEQ ID NO:7, and a light chain variable region comprising an amino acid sequence of SEQ ID NO:8.

In some embodiments, the anti-CD40 antibody, or antigen binding portion thereof, comprises a heavy chain comprising an amino acid sequence of SEQ ID NO:9, and a light chain comprising an amino acid sequence of SEQ ID NO:10.

In some embodiments, the anti-CD40 antibody or antigen-binding portion thereof is KPL-404.

In one aspect, the present invention provides a method of producing a pharmaceutical composition comprising an anti-CD40 antibody and a pharmaceutically acceptable carrier, the method comprising incubating a sample comprising the anti-CD40 antibody, or antigen- binding portion thereof, at a pH of about 3.6-3.9 during a viral inactivation step, thereby producing the pharmaceutical composition comprising the anti-CD40 antibody and the pharmaceutically acceptable carrier.

In some embodiments, the antibody preparation comprises charge species of the anti-CD40 antibody, wherein the charged species comprises at least about 40%, 41%, 42%, 43%, 44%, 45%, 46%, 47%, 48%, 49%, or 50% main species.

In some embodiments, the pharmaceutical composition comprises charge species of the anti-CD40 antibody, wherein the charged species comprises at least about 40%, 41%, 42%, 43%, 44%, 45%, 46%, 47%, 48%, 49%, or 50% main species.

### BRIEF DESCRIPTION OF THE DRAWING

FIG. 1 depicts an overview of an exemplary purification process.

FIG. 2 depicts an acid titration curve for KPL-404 under an extended virus inactivation hold.

FIG. 3 depicts the percentage of high molecular weight (HMW) protein aggregates in KPL-404 samples subjected to an extended virus inactivation hold (up to 300 minutes) under a pH of 3.5 and a pH of 3.6.

### DETAILED DESCRIPTION OF THE INVENTION

The present invention is based, at least, on the identification of surprising and unexpected virus inactivation and neutralization conditions during the purification process of a protein of interest, e.g., an anti-CD40 antibody, or antigen-binding portions thereof, which stabilizes the protein and/or minimizes formation of high molecular weight aggregates in the virus-inactivated protein preparation. In particular, the inventors of the present invention have surprisingly and unexpectedly discovered that despite pH ≤3.6 being the industry standard for the viral inactivation process, a substantial and unacceptable level of protein aggregates was observed during the viral inactivation step for an anti-CD40 antibody, e.g., KPL-404, at this pH range. Accordingly, a new and unexpected range for pH and/or hold time for viral inactivation was established to ensure that KPL-404 can be held for an extended period of time without compromising the final product quality.

The following description is presented to enable a person of ordinary skill in the art to make and use the various embodiments. Descriptions of specific methods, compositions, techniques, and applications are provided only as examples. Various modifications to the examples described herein will be readily apparent to those of ordinary skill in the art, and the general principles described herein may be applied to other examples and applications without departing from the spirit and scope of the various embodiments. Thus, the various embodiments are not intended to be limited to the examples described herein and shown, but are to be accorded the scope consistent with the claims.

Unless defined otherwise, all technical and scientific terms used herein have the same meaning as is commonly understood by one of ordinary skill in the art to which this application belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference. The headings provided herein are for convenience only and do not limit the application in any way. All patents, applications, published applications and other publications referred to herein are incorporated by reference in their entirety.

### I. Definition

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means ±3%. In certain embodiments, about means ±2%. In certain embodiments, about means ±1%. When about is present before a series of numbers or a range, it is understood that “about” can modify each of the numbers in the series or range.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein that includes modifications, such as deletions, additions, and substitutions (generally conservative in nature), to a native sequence, as long as the protein maintains the desired activity. These modifications may be deliberate, as through site-directed mutagenesis, or may be accidental, such as through mutations of hosts that produce the proteins or errors due to PCR amplification.

The term “antibody” includes an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CHI, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term antibody also includes chimeric antibodies, humanized antibodies, and antibodies of various species such as mouse, human, cynomolgus monkey, llama, camel, etc. The term also includes multivalent antibodies such as bivalent or tetravalent antibodies. A multivalent antibody includes, e.g., a single polypeptide chain comprising multiple antigen binding (CDR-containing) domains, as well as two or more polypeptide chains, each containing one or more antigen binding domains, such two or more polypeptide chains being associated with one another, e.g., through a hinge region capable of forming disulfide bond(s) or any other covalent or noncovalent interaction.

The term “antigen-binding portion” of an antibody (or “antibody portion”) includes fragments of an antibody, e.g., one or more antigen-binding domains, that retain the ability to specifically bind to an antigen (e.g., in the case of KPL-404, CD40). It has been shown that the antigen-binding function of an antibody can be performed by fragments of a full-length antibody. Examples of binding fragments encompassed within the term “antigen-binding portion” of an antibody include molecules comprising at least CDR1, CDR2, and CDR3 of a single domain antibody (sdAb), wherein the molecule is capable of binding to an antigen. The term antibody-binding portion also refers to molecules comprising at least CDR1, CDR2, and CDR3 of a heavy chain and CDR1, CDR2, and CDR3 of a light chain, wherein the molecule is capable of binding to an antigen. The term antibody-binding portion also includes fragments that are capable of binding an antigen, such as (i) a Fab fragment, a monovalent fragment comprising the VL, VH, CL and CH1 domains; (ii) a F(ab′)2 fragment, a bivalent fragment comprising two Fab fragments linked by a disulfide bridge at the hinge region; (iii) a Fab′ fragment, which can be formed by the reduction of F(ab′)2 fragment; (iv) a Fc fragment comprising the CH2 and CH3 region and part of the hinge region held together by one or more disulfides and noncovalent interactions; (v) a Fd fragment comprising the VH and CH1 domains; (vi) a Fv fragment comprising the VL and VH domains of a single arm of an antibody, (vii) a reduced IgG or half IgG; and (viii) a dAb fragment (Ward et al., (1989) Nature 341:544-546, the entire teaching of which is incorporated herein by reference), which comprises a VH domain. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883, the entire teachings of which are incorporated herein by reference). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al. (1993) Proc. Natl. Acad. Sci. USA 90:6444-6448; Poljak, R. J., et al. (1994) Structure 2:1121-1123, the entire teachings of which are incorporated herein by reference). Still further, an antibody or antigen-binding portion thereof may be part of a larger immunoadhesion molecule, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101, the entire teaching of which is incorporated herein by reference) and use of a cysteine residue, a marker peptide and a C-terminal polyhistidine tag to make bivalent and biotinylated scFv molecules (Kipriyanov, S. M., et al. (1994) Mol. Immunol. 31:1047-1058, the entire teaching of which is incorporated herein by reference). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. In one aspect, the antigen binding portions are complete domains or pairs of complete domains.

The term “human antibody” includes antibodies having variable and constant regions corresponding to human germline immunoglobulin sequences as described by Kabat et al. (See Kabat, et al. (1991) Sequences of proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), e.g., in the CDRs and in particular CDR3. The mutations can be introduced using the “selective mutagenesis approach.” The human antibody can have at least one position replaced with an amino acid residue, e.g., an activity enhancing amino acid residue which is not encoded by the human germline immunoglobulin sequence. The human antibody can have up to twenty positions replaced with amino acid residues which are not part of the human germline immunoglobulin sequence. In other embodiments, up to ten, up to five, up to three or up to two positions are replaced. In one embodiment, these replacements are within the CDR regions. However, the term “human antibody”, as used herein, is not intended to include antibodies in which CDR sequences derived from the germline of another mammalian species, such as a mouse, have been grafted onto human framework sequences.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see, e.g., Taylor, L. D., et al. (1992) Nucl. Acids Res. 20:6287-6295, the entire teaching of which is incorporated herein by reference) or antibodies prepared, expressed, created or isolated by any other means that involves splicing of human immunoglobulin gene sequences to other DNA sequences. Such recombinant human antibodies have variable and constant regions derived from human germline immunoglobulin sequences (see, Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or back-mutation or both.

An “isolated antibody”, as used herein, refers to an antibody that is substantially free of other antibodies having different antigenic specificities (e.g., an isolated antibody that specifically binds CD40 is substantially free of antibodies that specifically bind antigens other than CD40). An isolated antibody that specifically binds CD40 may, however, have cross-reactivity to other antigens, such as CD40 molecules from other species. Moreover, an isolated antibody may be substantially free of other cellular material and/or chemicals. A suitable anti-CD40 antibody is KPL-404.

The terms “Kabat numbering” “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242, the entire teachings of which are incorporated herein by reference). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

The term “product”, as used herein refers to a protein of interest, which may be present in the context of a sample comprising one or more variants and/or impurities, e.g., product-related substances, e.g., product aggregates, fragments or charged species, e.g., acidic or basic species, and/or process-related impurities, e.g., host cell proteins. In certain embodiments, the product, i.e., the protein of interest, is an antibody or antigen binding fragment thereof.

The term “fragments” as used herein refers to any truncated protein species from the protein of interest due to disruption of one or more bonds along the peptide backbone of a protein of interest, or dissociation of enzymatic and/or chemical modifications. For instance, antibody fragments include, but not limited to, Fab, F(ab′)2, Fab′, Fc, Fv, scFv, Fd, dAb, half antibody, or other compositions that contain a portion of the antibody molecule.

The terms “aggregates” or “high molecular weight aggregates” or “high molecular weight impurities”, as used herein, refer to the oligomerization of two or more individual molecules of protein of interest, including but not limiting to, protein dimers, trimers, tetramers, oligomers and other high molecular weight species.

The terms “charge variants” or “charged species”, as used herein, refer to the full complement of product with different charges. In certain embodiments, such variants can include product aggregates and/or product fragments, to the extent that such aggregation and/or fragmentation results in a product with charge variations as seen in an analytical technique used for that purpose. In certain embodiments, such variants refer to products with different modifications that give rise to charge heterogeneity. In monoclonal antibody preparations, charged variants, e.g., acidic species, or basic species, can be detected by charged based separation techniques such as isoelectric focusing (IEF) gel electrophoresis, capillary isoelectric focusing (cIEF) gel electrophoresis, cation exchange chromatography (CEX) and anion exchange chromatography (AEX).

As used herein, the term “acidic species” refers to the variants of a protein, e.g., an antibody or antigen-binding portion thereof, which are characterized by an overall acidic charge. Acidic species are variants with lower apparent pl when antibodies are analyzed using IEF based methods. When analyzed by chromatography-based methods, acidic species and basic species are defined based on their retention times relative to the main peak. Acidic species are the variants that elute earlier than the main peak from CEX or later then than the main peak from AEX.

The term “basic species”, as used herein, refers to the variants of a protein, e.g., an antibody or antigen-binding portion thereof, which are characterized by an overall basic charge. Basic species are variants with higher apparent pl when antibodies are analyzed using IEF based methods. When analyzed by chromatography-based methods, basic species are the variants that elute later than the main peak from CEX or earlier than the main peak from AEX.

The term “main species” as used herein, refers to the form of a protein, e.g., an antibody or antigen binding portion thereof, that elutes as the major peak on chromatograms, i.e., the majority species detected during fractionation of charged variants of a protein.

The term “process-related impurity,” as used herein, refers to impurities that are present in a composition comprising a protein but are not derived from the protein itself. Process-related impurities include, but are not limited to, host cell proteins (HCPs), host cell nucleic acids, e.g., DNA or RNA, chromatographic materials, and media components. Removal of process-related impurities, such host cell proteins, from the resulting protein product, e.g., an antibody or antigen-binding portion thereof, are desirable such that the resulting protein product would provide therapeutic benefits with higher potency, higher efficacy, or better stability without undesired effect.

The term “host cell proteins” (HCPs), as used herein, is intended to refer to non-target protein-related, proteinaceous impurities derived from host cells.

As used herein, the terms “CD40” refer to the well known gene and protein that is a member of the tumor necrosis factor (TNF)-receptor superfamily. The encoded protein is a receptor on antigen-presenting cells of the immune system and is essential for mediating a broad variety of immune and inflammatory responses including T cell-dependent immunoglobulin class switching, memory B cell development, and germinal center formation. CD40 is also known as Bp50, TNFRSF5, Tumor Necrosis Factor Receptor Superfamily Member 5, B Cell Surface Antigen CD40, CD40 antigen, CDw40, or CD40L receptor. The CD40 antigen is displayed on the surface of a variety of cell types, such as normal and neoplastic human B cells, dendritic cells, other antigen presenting cells (APCs), endothelial cells, monocytic cells, CD8+ T cells, epithelial cells, some epithelial carcinomas, and many solid tumors, including lung, breast, ovary, and colon cancers. Malignant B cells from several tumors of B-cell lineage express a high level of CD40 and appear to depend on CD40 signaling for survival and proliferation.

The term “CD40” includes human CD40, the amino acid sequence of which may be found in for example, GenBank Accession No. NP_001241.1 (SEQ ID NO:11), or NP_690593.1 (SEQ ID NO:12). The term “CD40” also includes cynomolgus CD40, mouse CD40, and rat CD40. The term “CD40” includes a wild type, a variant or an isoform of CD40 protein or a fragment or domain thereof. In certain embodiments, The CD40 protein may be coupled to a signal peptide sequence, and/or a protein tag.

As used herein, the term “KPL-404” refers to a monoclonal antibody designed to inhibit interaction of CD40 with CD154 (CD40 ligand), a well-known pathway that plays a critical role in regulating B cell proliferation, T cell activation, and antibody production (see PCT Publication No. WO2017040932, the entire contents of which, including the sequences described therein, are incorporated herein by reference). KPL-404 comprises a heavy chain comprising the sequence set forth as SEQ ID NO:9, and a light chain comprising the sequence set forth as SEQ ID NO:10. The heavy chain variable region of KPL-404 comprises the sequence set forth as SEQ ID NO:7, and the light chain variable region of KPL-404 comprises the sequence set forth as SEQ ID NO:8. The heavy chain variable region of KPL-404 comprises a CDR1 having the sequence set forth as SEQ ID NO:1, a CDR2 having the sequence set forth as SEQ ID NO:2, and a CDR3 having the sequence set forth as SEQ ID NO:3. The light chain variable region of KPL-404 comprises a CDR1 having the sequence set forth as SEQ ID NO:4, a CDR2 having the sequence set forth as SEQ ID NO:5 and a CDR3 having the sequence set forth as SEQ ID NO:6.

| | Chain, | | SEQ |
|---|---|---|---|
| Name | Region | Sequence | ID NO: |
| KPL- | Heavy chain, | YTFTNYWMH | 1 |
| 404 | CDR1 | | |
| KPL- | Heavy chain, | YINPSNDYTKYNQKFKD | 2 |
| 404 | CDR2 | | |
| KPL- | Heavy chain, | QGFPY | 3 |
| 404 | CDR3 | | |
| KPL- | Light chain, | SASSSVSYMH | 4 |
| 404 | CDR1 | | |
| KPL- | Light chain, | DTSKLAS | 5 |
| 404 | CDR2 | | |
| KPL- | Light chain, | HQLSSDPFT | 6 |
| 404 | CDR3 | | |
| KPL- | Heavy chain, | QVQLVQSGAEVKKPGASVKVSCKASGYTFTN | 7 |
| 404 | variable region | YWMHWVRQAPGQRLEWIGYINPSNDYTKYNQ | |
| | | KFKDRATLTADKSANTAYMELSSLRSEDTAV | |
| | | YYCARQGFPYWGQGTLVTVSS | |
| KPL- | Light chain, | EIVLTQSPATLSLSPGERATLSCSASSSVSY | 8 |
| 404 | variable region | MHWYQQKPGQAPRRWIYDTSKLASGVPARFS | |
| | | GSGSGTDYTLTISSLEPEDFAVYYCHQLSSD | |
| | | PFTFGGGTKVEIK | |
| KPL- | Heavy chain | QVQLVQSGAEVKKPGASVKVSCKASGYTFTN | 9 |
| 404 | (with IgG4 | YWMHWVRQAPGQRLEWIGYINPSNDYTKYNQ | |
| | constant | KFKDRATLTADKSANTAYMELSSLRSEDTAV | |
| | domain) | YYCARQGFPYWGQGTLVTVSSASTKGPSVFP | |
| | | LAPCSRSTSESTAALGCLVKDYFPEPVTVSW | |
| | | NSGALTSGVHTFPAVLQSSGLYSLSSVVTVP | |
| | | SSSLGTKTYTCNVDHKPSNTKVDKRVESKYG | |
| | | PPCPPCPAPEFLGGPSVFLFPPKPKDTLMIS | |
| | | RTPEVTCVVVDVSQEDPEVQFNWYVDGVEVH | |
| | | NAKTKPREEQFNSTYRVVSVLTVLHQDWLNG | |
| | | KEYKCKVSNKGLPSSIEKTISKAKGQPREPQ | |
| | | VYTLPPSQEEMTKNQVSLTCLVKGFYPSDIA | |
| | | VEWESNGQPENNYKTTPPVLDSDGSFFLYSR | |
| | | LTVDKSRWQEGNVFSCSVMHEALHNHYTQKS | |
| | | LSLSPGK | |
| KPL- | Light chain | EIVLTQSPATLSLSPGERATLSCSASSSVSY | 10 |
| 404 | (with kappa | MHWYQQKPGQAPRRWIYDTSKLASGVPARFS | |
| | constant | GSGSGTDYTLTISSLEPEDFAVYYCHQLSSD | |
| | domain) | PFTFGGGTKVEIKRTVAAPSVFIFPPSDEQL | |
| | | KSGTASVVCLLNNFYPREAKVQWKVDNALQS | |
| | | GNSQESVTEQDSKDSTYSLSSTLTLSKADYE | |
| | | KHKVYACEVTHQGLSSPVTKSFNRGEC | |

As used herein, the term “cell culture” refers to methods for generating and maintaining a population of host cells capable of producing a recombinant protein of interest, as well as the methods and techniques for optimizing the production and collection of the protein of interest. For example, once an expression vector has been incorporated into an appropriate host, the host can be maintained under conditions suitable for expression of the relevant nucleotide coding sequences, and the collection and purification of the desired recombinant protein.

As used herein, the term “downstream process technology” refers to one or more techniques used after the upstream process technologies to purify the protein of interest, e.g., antibody. For example, downstream process technology includes purification of the protein product using, for example, affinity chromatography, including Protein A affinity chromatography, ion exchange chromatography, such as anion or cation exchange chromatography, hydrophobic interaction chromatography, mixed-mode or multi-modal chromatography or displacement chromatography.

The term “virus inactivation” or “VI”, as used herein, refers to the treatment of a sample containing one or more viruses in a manner such that the one or more viruses are no longer able to replicate or are rendered inactive. Viral inactivation is intended to refer to a decrease in the number of viral particles in a particular sample, as well as a decrease in the activity, for example, but not limited to, the infectivity or ability to replicate, of viral particles in a particular sample. Virus inactivation may be achieved by physical means, e.g., heat, ultraviolet light, ultrasonic vibration, or using chemical means, e.g., pH change or addition of a chemical. Virus inactivation is typically a process step which is used during most protein purification processes, especially in case of purification of therapeutic proteins. It is understood that failure to detect one or more viruses in a sample using standard assays known in the art and those described herein, is indicative of complete inactivation of the one or more viruses following treatment of the sample. In some embodiments, the viral inactivation is achieved by a low pH treatment.

As used herein, the term “viral inactivation step” comprises (a) a virus inactivation period, i.e., a period immediately following the step of titrating a sample comprising a protein to be purified with acid to a pH sufficiently acidic to disrupt a viral envelope, during which time a substantial level of virus present is inactivated (e.g., about 15 minutes), and, optionally (b) a static holding period, i.e., a period following the virus inactivation period during which the sample is subjected to a static hold or an extended static hold.

The viral inactivation step is subsequently followed by neutralization with base to titrate the sample back up to a pH at which the protein is more stable and more suitable for the next purification step. The neutralized material is filtered for further processing.

The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “chromatography,” as used herein, refers to any kind of technique which separates the product of interest (e.g. a therapeutic protein or antibody) from contaminants and/or protein aggregates in a preparation.

The term “affinity chromatography” refers to a protein separation technique in which a target protein (e.g., a Fc region containing protein of interest or antibody) is specifically bound to a ligand (e.g. Protein A), which is typically immobilized onto a solid support (the ligand immobilized on the solid support is referred to herein as a “chromatography matrix”). The target protein generally retains its specific binding affinity for the ligand during the chromatographic steps, while other solutes and/or proteins in the mixture do not bind appreciably or specifically to the ligand. Binding of the target protein to the immobilized ligand allows impurities including contaminating proteins or protein impurities (e.g. HCPs) to be passed through the chromatography matrix while the target protein remains specifically bound to the immobilized ligand on the solid support material; however, some non-specific binding of the contaminating proteins onto the matrix is typically observed. The chromatography matrix is typically washed one or more times with a suitable wash buffer, in order to remove the non-specifically bound proteins (e.g., HCPs) and other impurities before eluting the bound protein from the matrix. The specifically bound protein of interest is subsequently eluted from the matrix using a suitable elution buffer which facilitates the separation of the protein of interest from the matrix. In embodiments according to the present invention, one or more intermediate wash steps are eliminated from such a process, without decreasing the purity of the eluted target protein. In other words, in some embodiments according to the present invention, a protein of interest is allowed to bind to a Protein A containing chromatography matrix and is subsequently eluted, without having the need for one or more intermediate wash steps; however, the purity of the protein of interest in the Protein A elution pool is not affected. In other embodiments, the number of intermediate wash steps are reduced compared to a process which would normally use a certain number of wash steps in order to achieve a certain level of purity of the protein of interest in the Protein A elution pool. In various embodiments according to the present invention, the level of host cell proteins is reduced in the Protein A elution pool, despite the elimination of or reduction in the number of intermediate wash steps.

The terms “ion-exchange” and “ion-exchange chromatography.” as used interchangeably herein, refer to the chromatographic process in which a solute or analyte of interest in a mixture, interacts with a charged compound linked (such as by covalent attachment) to a solid phase ion exchange material, such that the solute or analyte of interest interacts non-specifically with the charged compound more or less as compared to the solute impurities or contaminants in the mixture. The contaminating solutes in the mixture elute from a column of the ion exchange material faster or slower than the solute of interest or are bound to or excluded from the resin relative to the solute of interest. “Ion-exchange chromatography” includes cation exchange, anion exchange, and mixed mode ion exchange chromatography. For example, cation exchange chromatography can bind the target molecule (e.g., a Fc region containing target protein) followed by elution (cation exchange bind and elute chromatography) or can predominately bind the impurities while the target molecule “flows through” the column (cation exchange flow through chromatography). In case of anion exchange chromatography, the solid phase material can bind the target molecule (e.g. an Fc region containing target protein) followed by elution or can predominately bind the impurities while the target molecule “flows through” the column.

As used herein, the term “recombinant protein” refers to a protein produced as the result of the transcription and translation of a gene carried on a recombinant expression vector that has been introduced into a host cell. In certain embodiments, the recombinant protein is an antibody, e.g., a chimeric, humanized, or fully human antibody. In certain embodiments the recombinant protein is an antibody of an isotype selected from group consisting of: IgG (e.g., IgG1, IgG2, IgG3, IgG4), IgM, IgA1, IgA2, IgD, or IgE. In certain embodiments the antibody molecule is a full-length antibody (e.g., an IgG1 or IgG4 immunoglobulin) or alternatively the antibody can be a fragment (e.g., an Fc fragment or a Fab fragment).

### II. Methods of the Invention

The present invention is based, at least, on the identification of unexpected virus inactivation and neutralization conditions during the purification process of a protein of interest, e.g., an anti-CD40 antibody, or antigen-binding portions thereof, e.g., KPL-404, that stabilize the protein and/or reduces formation of high molecular weight aggregates in the virus-inactivated protein preparation.

In a typical purification process, once a protein of interest is expressed in cell culture, the cell culture media is subjected to a clarification step for removal of impurities and particulates, such as cells and cell debris. The clarified cell culture media containing the protein of interest is then subjected to one or more chromatography steps. In order to ensure safety of a protein of interest, especially in case of a therapeutic candidate, it is necessary to inactivate any enveloped viruses which may be present in a sample containing the protein of interest during the purification process.

Virus inactivation is generally performed after a chromatography step (e.g., affinity chromatography). For example, following a chromatography step, an elution pool containing the protein of interest is collected and subjected to a viral inactivation step for a period of time in order to achieve inactivation of enveloped viruses that may be present in the elution pool. In some embodiments, the chromatography step is an affinity chromatography step, such as Protein A affinity chromatography. Virus inactivation may be achieved by physical means, e.g., heat, ultraviolet light, ultrasonic vibration, or using chemical means, e.g., pH change or addition of a chemical. One or more of a variety of methods of viral reduction/inactivation can be used during purification. It is understood that failure to detect one or more viruses in a sample using standard assays known in the art and those described herein is indicative of complete inactivation of the one or more viruses following treatment of the sample.

Viral inactivation by low pH has been reliably demonstrated to inactivate large enveloped viruses (e.g., X-MuLV) in commercial purification processes. In the case of pH inactivation, operators titrate the pool to a pH sufficiently acidic to disrupt viral envelopes and subject the pool to a static hold, after which it is titrated back up to a pH at which the protein is more stable and which is more suitable for the next chromatography step. The pH conditions are selected as a balance between a low pH value that is sufficient to cause virus inactivation and a high enough value to avoid denaturation of the protein.

The inventors of the present invention have surprisingly and unexpectedly discovered that despite pH≤3.6 being the industry standard for the viral inactivation process, a substantial and unacceptable level of protein aggregates was observed during the viral inactivation step for an anti-CD40 antibody, e.g., KPL-404, at this pH range. Accordingly, a new and unexpected condition for viral inactivation was established for the purification process of KPL-404 that ensures sufficient viral inactivation while reducing and/or minimizing proten aggregation.

Accordingly, the present invention provides methods for producing a virus-inactivated antibody preparation comprising an anti-CD40 antibody, or antigen-binding portion thereof, having a reduced level of high molecular weight aggregates; methods for minimizing formation of high molecular weight aggregates in an antibody preparation comprising an anti-CD40 antibody, or antigen-binding portion thereof; methods for reducing formation of high molecular weight aggregates in an antibody preparation comprising an anti-CD40 antibody, or antigen-binding portion thereof; methods maximizing the level of antibody monomers in a virus-inactivated antibody preparation comprising an anti-CD40 antibody, or antigen-binding portion thereof, methods for stabilizing a virus-inactivated antibody preparation comprising an anti-CD40 antibody, or antigen-binding portion thereof; method of reducing the number and/or activity of viral particles in an antibody preparation comprising an anti-CD40 antibody, or antigen-binding portion thereof; and methods for producing a pharmaceutical composition comprising an anti-CD40 antibody, or antigen-binding portion thereof, and a pharmaceutically acceptable carrier. The methods comprise incubating a sample comprising the anti-CD40 antibody, or antigen-binding portion thereof, at a pH of about 3.6-3.9 during a viral inactivation step.

In some embodiments, the viral inactivation step comprises a virus inactivation period. In some embodiments, the viral inactivation step comprises a static holding period. In some embodiments, the viral inactivation step comprises a virus inactivation period and a static holding period.

In some embodiments, the antibody, or antigen binding portion thereof, comprises a heavy chain variable region comprising a CDR1 having the sequence set forth as SEQ ID NO:1, a CDR2 having the sequence set forth as SEQ ID NO:2, and a CDR3 having the sequence set forth as SEQ ID NO:3. In other embodiments, the antibody, or antigen binding portion thereof, comprises a light chain variable region comprising a CDR1 having the sequence set forth as SEQ ID NO:4, a CDR2 having the sequence set forth as SEQ ID NO:5 and a CDR3 having the sequence set forth as SEQ ID NO:6.

In some embodiments, the antibody, or antigen binding portion thereof, comprises a heavy chain variable region comprising the sequence set forth as SEQ ID NO:7, and a light chain variable region comprising the sequence set forth as SEQ ID NO:8.

In some embodiments, the antibody, or antigen binding portion thereof, comprises a heavy chain comprising the sequence set forth as SEQ ID NO:9, and a light chain comprising the sequence set forth as SEQ ID NO:10.

In some embodiments, the anti-CD40 antibody, or antigen binding portion thereof, is KPL-404, or an antigen binding portion thereof.

In some embodiments, the sample is subject to an affinity chromatography column, e.g., a Protein A chromatography column, prior to the viral inactivation step. In some embodiments, the sample is incubated at a pH of about 3.6-3.9, about 3.6-3.7, about 3.7-3.8, about 3.8-3.9, about 3.6-3.8 or about 3.7-3.9 during the viral inactivation step, e.g., during the static holding period or during an extended static holding period. In some embodiments, the sample has a pH of about 3.6-3.9, about 3.6-3.7, about 3.7-3.8, about 3.8-3.9, about 3.6-3.8 or about 3.7-3.9. In some embodiments, the sample is incubated at a pH of about 3.6, about 3.7, about 3.8, or about 3.9 during the viral inactivation step. In some embodiments, the sample has a pH of about 3.6, about 3.7, about 3.8, or about 3.9.

In some embodiments, the sample is incubated during the viral inactivation step for about 10-360 minutes. In some embodiments, the sample is incubated during the viral inactivation step for about 30-120 minutes, e.g., about 30-60 minutes, about 60-90 minutes, about 60-120 minutes, or about 90-120 minutes. In some embodiments, the sample is incubated during the viral inactivation step for about 60-120 minutes. In some embodiments, the sample is incubated during the viral inactivation step for about 10-50 minutes, about 20-40 minutes, about 20-60 minutes, about 30-70 minutes, about 40-80 minutes, about 50-70 minutes, about 50-90 minutes, about 60-100 minutes, about 70-110 minutes, about 80-100 minutes, about 80-120 minutes, about 60-120 minutes, about 90-130 minutes, about 100-140 minutes, about 110-130 minutes, about 110-150 minutes, about 120-160 minutes, about 130-170 minutes, about 140-180 minutes, about 150-190 minutes, about 160-200 minutes, about 170-190 minutes, about 170-210 minutes, about 180-220 minutes, about 200-240 minutes, about 220-260 minutes, about 230-250 minutes, about 240-280 minutes, about 280-320 minutes, about 290-310 minutes, or about 320-360 minutes.

In some embodiments, the sample is incubated during the viral inactivation step for at least about 15 minutes, at least about 30 minutes, at least about 60 minutes, at least about 90 minutes, or at least about 120 minutes. In some embodiments, the sample is incubated during the viral inactivation step for about 15 minutes, about 20 minutes, about 30 minutes, about 40 minutes, about 50 minutes, about 60 minutes, about 70 minutes, about 80 minutes, about 90 minutes, about 100 minutes, about 110 minutes, about 120 minutes, about 130 minutes, about 140 minutes, about 150 minutes, about 160 minutes, about 170 minutes, about 180 minutes, about 190 minutes, about 200 minutes, about 210 minutes, about 220 minutes, about 230 minutes, about 240 minutes, about 250 minutes, about 260 minutes, about 270 minutes, about 280 minutes, about 290 minutes, about 300 minutes, about 310 minutes, about 320 minutes, about 330 minutes, about 340 minutes, about 350 minutes, or about 360 minutes.

In some embodiments, the sample is incubated during the viral inactivation step at a temperature of about 4° C.-37° C., about 10° C.-37° C., about 10° C.-20° C., about 15° C.-30° C., about 15° C.-25° C., about 15° C.-37° C., or about 13° C.-25° C.

In some embodiments, the sample is incubated during the viral inactivation step at a pH of about 3.6-3.9 for about 30-120 minutes. In some embodiments, the sample is incubated during the viral inactivation step at a pH of about 3.6-3.9 for about 60-120 minutes.

In some embodiments, the sample is incubated during the viral inactivation step at a pH of about 3.7-3.9 for about 30-120 minutes. In some embodiments, the sample is incubated during the viral inactivation step at a pH of about 3.7-3.9 for about 60-120 minutes. In other embodiments, the sample is incubated during the viral inactivation step at a pH of about 3.7-3.9 at a temperature of about 15° C.-25° C. for about 50-70 minutes.

In some embodiments, the sample is incubated during the viral inactivation step at a pH of about 3.6-3.8 for about 30-120 minutes. In some embodiments, the sample is incubated during the viral inactivation step at a pH of about 3.6-3.8 for about 60-120 minutes.

In some embodiments, the sample is incubated during the viral inactivation step at a pH of about 3.6-3.9 at a temperature of about 15° C.-25° C. for about 30-120 minutes. In some embodiments, the sample is incubated during the viral inactivation step at a pH of about 3.7-3.9 at a temperature of about 15° C.-25° C. for about 30-120 minutes. In other embodiments, the sample is incubated during the viral inactivation step at a pH of about 3.6-3.8 at a temperature of about 15° C.-25° C. for about 30-120 minutes.

In some embodiments, the sample is incubated during the viral inactivation step at a pH of about 3.6-3.9 at a temperature of about 15° C.-25° C. for about 60-120 minutes. In some embodiments, the sample is incubated during the viral inactivation step at a pH of about 3.7-3.9 at a temperature of about 15° C.-25° C. for about 60-120 minutes. In other embodiments, the sample is incubated during the viral inactivation step at a pH of about 3.6-3.8 at a temperature of about 15° C.-25° C. for about 60-120 minutes.

In some embodiments, the sample is incubated during the viral inactivation step at a pH of about 3.7 for about 30-120 minutes. In some embodiments, the sample is incubated during the viral inactivation step at a pH of about 3.7 for about 60-120 minutes.

In some embodiments, the sample is incubated during the viral inactivation step at a pH of about 3.7 at a temperature of about 15° C.-25° C. for about 30-120 minutes. In some embodiments, the sample is incubated during the viral inactivation step at a pH of about 3.7 at a temperature of about 15° C.-25° C. for about 60-120 minutes. In some embodiments, the sample incubated during the viral inactivation step at a pH of about 3.7 for about 120 minutes.

In some embodiments, the sample is incubated during the viral inactivation step at a pH of about 3.7 at a temperature of about 25° C. for about 30-120 minutes. In some embodiments, the sample is incubated during the viral inactivation step at a pH of about 3.7 at a temperature of about 25° C. for about 120 minutes.

In some embodiments, the sample is incubated during the viral inactivation step at a pH of about 3.7-3.8 at a temperature of about 13° C.-25° C. for about 30-120 minutes.

In some embodiments, the sample is incubated during the viral inactivation step at a pH of about 3.7-3.8 at a temperature of about 15° C.-25° C. for about 30-120 minutes.

In some embodiments, the sample is incubated during the viral inactivation step at a pH of about 3.7-3.8 at a temperature of about 15° C.-25° C. for about 60-120 minutes.

In other embodiments, the sample is incubated during the viral inactivation step at a pH of about 3.8 at a temperature of about 15° C.-25° C. for about 60 minutes.

The virus-inactivated antibody preparation has a reduced level of high molecular weight aggregates. In some embodiments, the virus-inactivated antibody preparation comprises less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% high molecular weight aggregates, and ranges within one or more of the preceding.

In some embodiments, the virus-inactivated antibody preparation comprises less than about 10% high molecular weight aggregates. In some embodiments, the virus-inactivated antibody preparation comprises less than about 5% high molecular weight aggregates. In some embodiments, the virus-inactivated antibody preparation comprises less than about 2% high molecular weight aggregates. In some embodiments, the virus-inactivated antibody preparation comprises less than about 1% high molecular weight aggregates. In some embodiments, the virus-inactivated antibody preparation comprises less than about 0.5% high molecular weight aggregates.

In some embodiments, the virus-inactivated antibody preparation comprises about 0.1-10%, about 0.1-9%, about 0.1-8%, about 0.1-7%, about 0.1-6%, about 0.1-5%, about 0.1-4%, about 0.1%-3%, about 0.1%-2%, or about 0.1-1% high molecular weight aggregates, and ranges within one or more of the preceding. In some embodiments, the virus-inactivated antibody preparation comprises about 0.1-2% high molecular weight aggregates. In some embodiments, the virus-inactivated antibody preparation comprises about 0.1-1% high molecular weight aggregates.

The levels of high molecular weight aggregates can be analyzed by any methods known in the art. In certain embodiments, the level of aggregate is measured using a size exclusion chromatographic (SEC) method. Any additional technique, such as mass spectroscopy, can also be used for assaying size variants.

In one embodiment, the virus-inactivated antibody preparation comprises charge species of the antibody comprising at least about 40%, 41%, 42%, 43%, 44%, 45%, 46%, 47%, 48%, 49%, 50% or 51% main species. In other embodiments, the virus-inactivated antibody preparation comprises charged species of the antibody comprising about 40-55%, 40-54%, 40-53%, 40-52%, 40-51% about 40-50%, about 40-49%, about 40-48%, about 40-47%, about 40-46%, about 40-45%, about 46-51% or about 40-44% main species.

The levels of charged species can be analyzed by any methods known in the art. In certain embodiments, the level of a charge species is measured by charged based separation techniques such as a isoelectric focusing (IEF) gel electrophoresis method, e.g., capillary IEF (cIEF), and imaged cIEF (IcIEF). Any additional technique, such as cation exchange chromatography (CEX) and anion exchange chromatography (AEX), can also be used for assaying charge variants.

In certain embodiments, the virus-inactivated antibody preparation can be further subjected to one or more additional purification steps, e.g., anion exchange chromatography, or cation exchange chromatography, or mixed mode chromatography columns. Further purification procedures may also include virus filtration and ultrafiltration/diafiltration.

Following the polishing chromatography steps, the eluate pool may be subjected to a nanofiltration step. In an embodiment, the nanofiltration step is accomplished via one or more nanofilters or viral filters. The filters may be any known in the art to be useful for this purpose and may include, for example, EMD Millipore Viresolve VPro, Viresolve NFP, Viresolve NFR, or Planova 15N, 20N, and 35N virus removal filters from Asashi Kasei Pharma. In certain embodiments, the nanofiltration filter has a mean pore size of between about 15 nm and about 200 nm. One of skill in the art will understand that the selection of types and numbers of filters will be dependent on the volume of sample being processed and the desired filtration performance.

Ultrafiltration and diafiltration steps may also be included to further concentrate and formulate the protein of interest, e.g., an CD-40 antibody, or antigen binding portion thereof, such as KPL-404. The nanofiltration step may be followed by ultrafiltration and diafiltration to achieve the targeted drug substrance concentration and buffer condition before formulation. Ultrafiltration is generally considered to mean filtration using filters with a molecular weight cut-off of about 10 kDa. By employing filters having such small pore size, the volume of the sample can be reduced through permeation of the sample buffer through the filter membrane pores while proteins, such as antibodies, are retained above the membrane surface. Diafiltration is a method of using membrane filters to remove and exchange salts, sugars, and non-aqueous solvents, to separate free from bound species, to remove low molecular-weight species, and/or to cause the rapid change of ionic and/or pH environments. Microsolutes are removed most efficiently by adding solvent to the solution being diafiltered at a rate approximately equal to the permeate flow rate. This washes away microspecies from the solution at a constant volume, effectively purifying the retained protein of interest. In certain embodiments of the present invention, a diafiltration step is employed to exchange the various buffers used in connection with the instant invention, optionally prior to further chromatography or other purification steps, as well as to remove impurities from the protein preparations. One of ordinary skill in the art can select appropriate membrane filter device for the UF/DF operation. Examples of membrane cassettes suitable for the present invention include, but not limited to, Sartorius Vivaspin, Pellicon 2 or Pellicon 3 cassettes with 10 kD, 30 kD or 50 kD membranes from EMD Millipore, Kvick 10 kD, 30 kD or 50 kD membrane cassettes from GE Healthcare, and Centramate or Centrasette 10 kD, 30 kD or 50 kD cassettes from Pall Corporation. Upon completion of the diafiltration step, the protein concentration of the solution can be adjusted to with the diafiltration buffer to a desired final concentration.

### III. Compositions of the Invention

The present invention includes, in one aspect, compositions comprising a virus-inactivated eluate collected from an affinity chromatography column (e.g., a protein A column) comprising a protein, having a reduced level of high molecular weight aggregates. In one embodiment, the protein is an anti-CD40 antibody, or antigen-binding portion thereof. In another embodiment, the anti-CD40 antibody is KPL-404.

In some embodiments, the composition comprises a virus-inactivated eluate that has been subjected to an affinity chromatography column, wherein the eluate comprises less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% high molecular weight aggregates, and ranges within one or more of the preceding.

In some embodiments, the virus-inactivated eluate comprises less than about 10% high molecular weight aggregates. In some embodiments, the virus-inactivated eluate comprises less than about 5% high molecular weight aggregates. In some embodiments, the virus-inactivated eluate comprises less than about 2% high molecular weight aggregates. In some embodiments, the virus-inactivated eluate comprises less than about 1% high molecular weight aggregates. In some embodiments, the virus-inactivated eluate comprises less than about 0.5% high molecular weight aggregates.

In some embodiments, the virus-inactivated eluate comprises about 0.1-10%, about 0.1-9%, about 0.1-8%, about 0.1-7%, about 0.1-6%, about 0.1-5%, about 0.1-4%, about 0.1%-3%, about 0.1%-2%, or about 0.1-1% high molecular weight aggregates, and ranges within one or more of the preceding. In some embodiments, the virus-inactivated eluate comprises about 0.1-2% high molecular weight aggregates.

In some embodiments, the eluate that has been subjected to an affinity chromatography column, e.g., a protein A chromatography column, has been additionally subjected to a viral inactivation step and has a pH of about 3.6-3.7, about 3.7-3.8, about 3.8-3.9, about 3.6-3.8, about 3.6-3.9, about 3.7-3.9, e.g., during a static holding period or during an extended static holding period. In some embodiments, the eluate has a pH of about 3.6-3.7, about 3.7-3.8, about 3.8-3.9, about 3.6-3.8, about 3.6-3.9, about 3.7-3.9, during the viral inactivation step. In some embodiments, the eluate that has been subjected to an affinity chromatography column has a pH of about 3.6, about 3.7, about 3.8, or about 3.9 during the viral inactivation step. In some embodiments, the eluate has a pH of about 3.6, about 3.7, about 3.8, or about 3.9.

In some embodiments, the eluate during the viral inactivation step has a temperature of about 4° C.-37° C., about 10° C.-37° C., about 10° C.-20° C., about 15° C.-30° C., about 15° C.-25° C., about 15° C.-37° C., or about 13° C.-25° C.

In some embodiments, the eluate has a pH of about 3.6-3.9 and a temperature of about 15° C.-25° C. In some embodiments, the eluate has a pH of about 3.6-3.8 and a temperature of about 15° C.-25° C. In some embodiments, the eluate has a pH of about 3.7-3.9 and a temperature of about 15° C.-25° C. In some embodiments, the eluate has a pH of about 3.7-3.9 and a temperature of about 13° C.-25° C.

In some embodiments, the eluate has a pH of about 3.7 and a temperature of about 15° C.-25° C. In some embodiments, the eluate has a pH of about 3.8 and a temperature of about 15° C.-25° C.

In some embodiments, the protein in the virus-inactivated eluate collected from an affinity chromatography column (e.g., a protein A column) comprises an antibody, or antigen-binding portion thereof, having a reduced level of high molecular weight aggregates. For example, the antibody, or antigen binding portion thereof may be an anti-CD40 antibody, or antigen binding portion thereof, such as KPL-404, or an antigen binding portion thereof.

In some embodiments, the antibody, or antigen binding portion thereof, comprises a heavy chain variable region comprising a CDR1 having the sequence set forth as SEQ ID NO:1, a CDR2 having the sequence set forth as SEQ ID NO:2, and a CDR3 having the sequence set forth as SEQ ID NO:3. In other embodiments, the antibody, or antigen binding portion thereof, comprises a light chain variable region comprising a CDR1 having the sequence set forth as SEQ ID NO:4, a CDR2 having the sequence set forth as SEQ ID NO:5, and a CDR3 having the sequence set forth as SEQ ID NO:6.

In some embodiments, the antibody, or antigen binding portion thereof, comprises a heavy chain variable region comprising the sequence set forth as SEQ ID NO:7, and a light chain variable region comprising the sequence set forth as SEQ ID NO:8.

In some embodiments, the antibody, or antigen binding portion thereof, comprises a heavy chain comprising the sequence set forth as SEQ ID NO:9, and a light chain comprising the sequence set forth as SEQ ID NO:10.

In certain embodiments, the antibody, or antigen binding portion thereof, that can be used in the compositions of the present disclosure can be generated by a variety of techniques, including immunization of an animal with the antigen of interest followed by conventional monoclonal antibody methodologies e.g., the standard somatic cell hybridization technique of Kohler and Milstein (1975) Nature 256:495. Somatic cell hybridization procedures can be used. In principle, other techniques for producing monoclonal antibody can be employed as well, including viral or oncogenic transformation of B lymphocytes.

One exemplary animal system for preparing hybridomas is the murine system. Hybridoma production is a very well-established procedure. Immunization protocols and techniques for isolation of immunized splenocytes for fusion are known in the art. Fusion partners (e.g., murine myeloma cells) and fusion procedures are also known.

An antibody used in the compositions of the invention can be a human, a chimeric, or a humanized antibody.

In one non-limiting embodiment, the antibodies to be used in the compositions of the invention are human monoclonal antibodies. Such human monoclonal antibodies can be generated using transgenic or transchromosomic mice carrying parts of the human immune system rather than the mouse system. These transgenic and transchromosomic mice include mice referred to herein as the HuMAb Mouse® (Medarex, Inc.), KM Mouse® (Medarex, Inc.), and XenoMouse® (Amgen). The antibodies, or antigen-binding portions thereof, used in the compositions of the invention can also be produced using the methods described in U.S. Pat. No. 6,090,382, the entire contents of which is expressly incorporated herein by reference.

Moreover, alternative transchromosomic animal systems expressing human immunoglobulin genes are available in the art and can be used to raise antibodies of the disclosure. For example, mice carrying both a human heavy chain transchromosome and a human light chain transchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (e.g., Kuroiwa et al. (2002) Nature Biotechnology 20:889-894 and PCT application No. WO 2002/092812) and can be used to raise antibodies of this disclosure.

Recombinant human antibodies to be used in the compositions of the invention can be isolated by screening of a recombinant combinatorial antibody library, e.g., a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SurfZAP™ phage display kit, catalog no. 240612, the entire teachings of which are incorporated herein), examples of methods and reagents particularly amenable for use in generating and screening antibody display libraries can be found in, e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibody Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; and Barbas et al. (1991) PNAS 88:7978-7982; the entire teachings of which are incorporated herein.

Human monoclonal antibodies to be used in the compositions of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In certain embodiments, the human antibodies to be used in the compositions of the invention are anti-CD40 antibodies and antibody portions thereof, anti-CD40-related antibodies and antibody portions, and human antibodies and antibody portions with equivalent properties to anti-CD40 antibodies, such as high affinity binding to CD40 with low dissociation kinetics and high neutralizing capacity. In one embodiment, an anti-CD40 antibody to be used in the compositions of the invention binds to the same epitope on CD40 as KPL-404. In another embodiment, an anti-CD40 antibody to be used in the compositions of the invention competitively inhibits binding of KPL-404 to CD40 under physiological conditions. In one embodiment, the compositions of the invention comprise KPL-404, or an antigen binding portion thereof.

Antibodies or antigen binding portion thereof to be used in the compositions of the invention can be altered, wherein the constant region of the antibody is modified to reduce at least one constant region-mediated biological effector function relative to an unmodified antibody. To modify an antibody of the invention such that it exhibits reduced binding to the Fc receptor, the immunoglobulin constant region can be mutated at particular regions necessary for Fc receptor (FcR) interactions (see, e.g., Canfield and Morrison (1991) J. Exp. Med. 173:1483-1491; and Lund et al. (1991) J. of Immunol. 147:2657-2662, the entire teachings of which are incorporated herein). Reduction in FcR binding ability of the antibody may also reduce other effector functions which rely on FcR interactions, such as opsonization and phagocytosis and antigen-dependent cellular cytotoxicity.

In another aspect, the present invention provides compositions comprising an antibody preparation, e.g., an anti-CD40 antibody, or antigen-binding portion thereof, e.g., KPL-404. The anti-CD40 antibody, or antigen-binding portion thereof, is prepared based on the methods of the present invention, i.e., the sample comprising the anti-CD40 antibody, or antigen-binding portion thereof, has undergone a viral inactivation step that stabilizes the antibody and/or reduces formation of high molecular weight aggregates in the virus-inactivated antibody preparation.

In some embodiments, the sample comprising the anti-CD40 antibody, or antigen-binding portion thereof, has been incubated at a pH of about 3.6-3.9 during the viral inactivation step.

In some embodiments, the composition compirising the anti-CD40 antibody, or antigen-binding portion thereof, has a reduced level of high molecular weight aggregates.

In some embodiments, the composition compirising the anti-CD40 antibody, or antigen-binding portion thereof, comprises less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% high molecular weight aggregates, and ranges within one or more of the preceding.

In some embodiments, the composition compirising the anti-CD40 antibody, or antigen-binding portion thereof, comprises less than about 10% high molecular weight aggregates. In some embodiments, the composition compirising the anti-CD40 antibody, or antigen-binding portion thereof, comprises less than about 5% high molecular weight aggregates. In some embodiments, the composition compirising the anti-CD40 antibody, or antigen-binding portion thereof, comprises less than about 2% high molecular weight aggregates. In some embodiments, the composition compirising the anti-CD40 antibody, or antigen-binding portion thereof, comprises less than about 1% high molecular weight aggregates. In some embodiments, the composition compirising the anti-CD40 antibody, or antigen-binding portion thereof, comprises less than about 0.5% high molecular weight aggregates.

In some embodiments, the composition compirising the anti-CD40 antibody, or antigen-binding portion thereof, comprises about 0.1-10%, about 0.1-9%, about 0.1-8%, about 0.1-7%, about 0.1-6%, about 0.1-5%, about 0.1-4%, about 0.1%-3%, about 0.1%-2%, or about 0.1-1% high molecular weight aggregates, and ranges within one or more of the preceding. In some embodiments, the composition compirising the anti-CD40 antibody, or antigen-binding portion thereof, comprises about 0.1-2% high molecular weight aggregates. In some embodiments, the composition compirising the anti-CD40 antibody, or antigen-binding portion thereof, comprises about 0.1-1% high molecular weight aggregates.

In some embodiments, the anti-CD40 antibody, or antigen binding portion thereof, comprises a heavy chain variable region comprising a CDR1 having the sequence set forth as SEQ ID NO:1, a CDR2 having the sequence set forth as SEQ ID NO:2, and a CDR3 having the sequence set forth as SEQ ID NO:3. In other embodiments, the anti-CD40 antibody, or antigen binding portion thereof, comprises a light chain variable region comprising a CDR1 having the sequence set forth as SEQ ID NO:4, a CDR2 having the sequence set forth as SEQ ID NO:5, and a CDR3 having the sequence set forth as SEQ ID NO:6.

In some embodiments, the anti-CD40 antibody, or antigen binding portion thereof, comprises a heavy chain variable region comprising the sequence set forth as SEQ ID NO:7, and a light chain variable region comprising the sequence set forth as SEQ ID NO:8.

In some embodiments, the anti-CD40 antibody, or antigen binding portion thereof, comprises a heavy chain comprising the sequence set forth as SEQ ID NO:9, and a light chain comprising the sequence set forth as SEQ ID NO:10.

In some embodiments, the anti-CD40 antibody, or antigen binding portion thereof, comprises KPL-404, or an antigen binding portion thereof.

### IV. Methods of Treatment Using the Compositions of the Invention

The compositions of the invention comprising an antibody preparation that has undergone a viral inactivation step at a pH of about 3.6-3.9 may be used to treat any disorder in a subject for which the therapeutic protein comprised in the composition is appropriate for treating. In some embodiments, the antibody preparation has undergone a viral inactivation step at a pH of about 3.6-3.7, about 3.7-3.8, about 3.8-3.9, about 3.6-3.8, about 3.7-3.9. In some embodiments, the antibody preparation has undergone a viral inactivation step at a pH of about 3.6, about 3.7, about 3.8, or about 3.9. In one embodiment, the antibody is an anti-CD40 antibody, or antigen-binding portion thereof. In another embodiment, the anti-CD40 antibody is KPL-404.

A “disorder” is any condition that would benefit from treatment with the protein. This includes chronic and acute disorders or diseases including those pathological conditions which predispose the subject to the disorder in question. In the case of an anti-CD40 antibody, or antigen binding portion thereof, such as KPL-404, a therapeutically effective amount of the composition may be administered to treat a CD40-associated disorder.

As used herein, the term “CD40-associated disease or disorder” is intended to include diseases and other disorders in which the presence of CD40 in a subject suffering from the disorder has been shown to be or is suspected of being either responsible for the pathophysiology of the disorder or a factor that contributes to a worsening of the disorder. Accordingly, a CD40-associated disorder is a disorder in which inhibition of CD40 activity is expected to alleviate the symptoms and/or progression of the disorder. The compositions of the invention can be used in treating any CD40-associated diseases or disorders known in the art including, but not limited to, autoimmune diseases, immunologic disorders, inflammatory disorders, and cancer.

As used herein, the term “subject” is intended to include living organisms. Examples of subjects include mammals, e.g., humans, dogs, cows, horses, pigs, sheep, goats, cats, mice, rabbits, rats, and transgenic non-human animals. In specific embodiments of the invention, the subject is a human.

As used herein, the term “treatment” or “treat” refers to both therapeutic treatment and prophylactic or preventative measures. Those in need of treatment include those already with the disorder, as well as those in which the disorder is to be prevented.

The composition can be administered by a variety of methods known in the art. Exemplary routes/modes of administration include intravenous, intramuscular, intranasal, oral, topical or subcutaneous delivery. As will be appreciated by the skilled artisan, the route and/or mode of administration will vary depending upon the desired results.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. In certain embodiments it is especially advantageous to formulate parenteral compositions in dosage unit form for case of administration and uniformity of dosage. Dosage unit form as used herein refers to physically discrete units suited as unitary dosages for the mammalian subjects to be treated; each unit comprising a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of developing such an active compound for the treatment of a particular disease or disorder in individuals.

It is to be noted that a dosing regimen of a therapeutic antibody administered to a subject may vary depending on the characteristics of the specific antibody (e.g., binding affinity, and pharmacokinetic and pharmacodynamic properties) and with the type and severity of the condition to be alleviated. It is to be further understood that for any particular subject, specific dosage regimens should be adjusted over time according to the individual need, the result to be achieved, and the professional judgment of the person administering or supervising the administration of the compositions.

### VII. Pharmaceutical Formulations

The present invention further provides preparations and formulations comprising the compositions of the invention. It should be understood that the compositions comprising an antibody preparation that has undergone a viral inactivation step at a pH of about 3.6-3.9 may be formulated or prepared as described below. In one embodiment, the antibody is an anti-CD40 antibody, or antigen-binding portion thereof. In another embodiment, the anti-CD40 antibody is KPL-404.

In certain embodiments, the compositions of the invention may be formulated with a pharmaceutically acceptable carrier as pharmaceutical (therapeutic) compositions, and may be administered by a variety of methods known in the art. As will be appreciated by the skilled artisan, the formulation and route and/or mode of administration will vary depending upon the physical and pharmacologic properties of the therapeutic antibody and the desired results.

The term “pharmaceutically acceptable carrier” means one or more non-toxic materials that do not interfere with the effectiveness of the biological activity of the active ingredients.

The compositions of the invention are present in a form acceptable for therapeutic uses. In one embodiment, a formulation of the compositions of the invention is a liquid formulation. In another embodiment, a formulation of the compositions of the invention is a lyophilized formulation. In a further embodiment, a formulation of the compositions of the invention is a reconstituted liquid formulation. In one embodiment, a formulation of the compositions of the invention is a stable liquid formulation. In one embodiment, a liquid formulation of the compositions of the invention is an aqueous formulation. In another embodiment, the liquid formulation is non-aqueous. In a specific embodiment, a liquid formulation of the compositions of the invention is an aqueous formulation wherein the aqueous carrier is distilled water.

The compositions of the invention can be formulated for particular routes of administration, such as oral, nasal, pulmonary, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. By way of example, in certain embodiments, the antibodies (including antibody fragments) are formulated for intravenous administration. In certain other embodiments, the antibodies (including antibody fragments) are formulated for local delivery to the cardiovascular system, for example, via catheter, stent, wire, intramyocardial delivery, intrapericardial delivery, or intraendocardial delivery. In a particular embodiment, the composition comprises an anti-CD40 antibody such as KPL-404 and is formulated for subcutaneous administration.

Formulations of the compositions of the invention which are suitable for topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required (U.S. Pat. Nos. 7,378,110; 7,258,873; 7,135,180; 7,923,029; and US Publication No. 20040042972).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Actual dosage levels of the active ingredients in the pharmaceutical compositions of the compositions of the invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed,, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The present invention is further illustrated by the following examples which should not be construed as limiting in any way.

### EXAMPLES

### Example 1: Downstream Process for Production of an Anti-CD40 Antibody

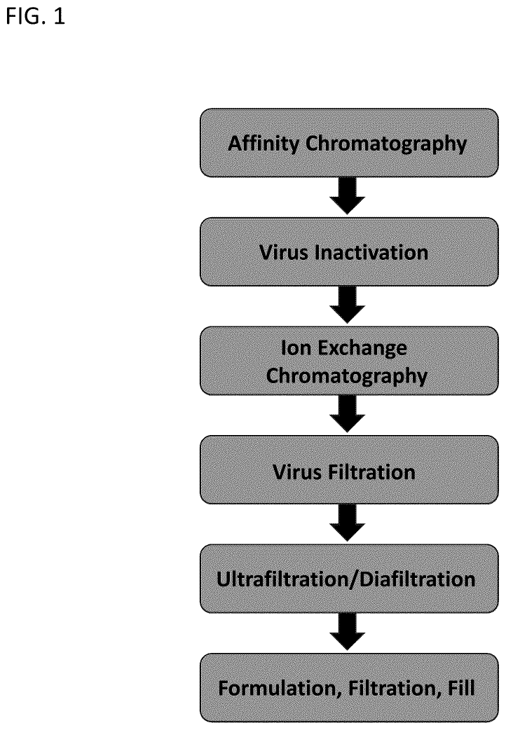

KPL-404 is a monoclonal antibody that binds to CD40 and blocks CD154-mediated activation of B cells. KPL-404 is derived from a stable CHO cell line and is comprised of two light chains and two heavy chains. The downstream manufacturing process consisted of a Protein A capture step, a low-pH hold to neutralize viruses, ion-exchange chromatography steps, nanofiltration, and ultrafiltration/diafiltration (UFDF) prior to final formulation, filtration and fill (FIG. 1).

This Example describes the response of KPL-404 to variations in process parameters, in particular, the pH value during the viral inactivation step, to investigate the impact on product quality.

### Run 1

Studies were performed to establish a risk profile of changes in product quality as a consequence of an unintended variation in pH during a viral inactivation (VI) hold. During the initial set of robustness studies, the first extended viral inactivation (VI) hold experiment was conducted at pH 3.27 and spanned for a total of 360 minutes. To ensure that a point of failure, if any, could be established within this timeframe with accuracy, samples of that pool were pulled at regular intervals between 0 and 360 minutes, neutralized individually to pH 5.0, filtered, and retained. If testing were to yield unacceptable product quality (PQ) data for the 360-minute pool, these intermediate pools could be submitted for testing to determine when degradation occurred. A total of five timepoints were taken beginning at 120 minutes. Samples were submitted for size exclusion chromatography (SEC), imaged capillary isoelectric focusing (iCIEF) and non-reduced capillary electrophoresis sodium dodecyl sulfate (NR-CESDS) analysis.

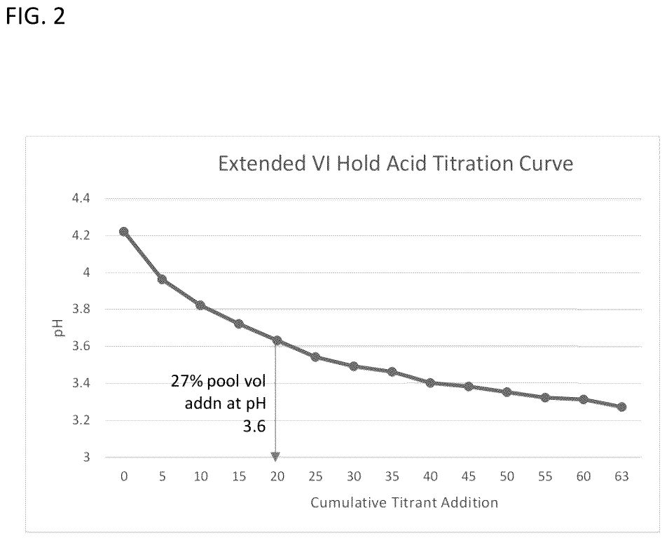

Extended hold parameters and the acid titration curve were presented in Table 1 and FIG. 2 while analytical data from the extended hold, the VI hold for the main material generation (MG) processing pool, and a reference VI pool for comparison was presented in Table 2.

TABLE 1
Extended Hold Parameters

| | |
|---|---|
| Starting pH | 4.22 |
| Titrant Addition Volume | 84% |
| (0.5M Acetic Acid) | |
| Ending pH | 3.27 |

TABLE 2
VI Hold Data

| | | | | IcIEF |
|---|---|---|---|---|
| | | SEC | NR-CESDS | (main/ |
| | | (% monomer/ | (% monomer/ | acidic/ |
| | | % aggregate) | % impurity) | basic) |
| pH 3.6, 90 | KCD21002A | 99.5/0.5 | 98.9/0.6 | 50.7/32.3/ |
| min | | | | 17.0 |
| pH 3.5, 90 | MG Run | 95.0/5.0 | 98.5/1.5 | 48.0/32.0/ |
| min | | | | 20.1 |
| pH 3.3, 1-6 | Ext Hold | 44.4/55.2 | 98.5/1.4 | 30.0/20.9/ |
| hrs | 120 min | | | 49.1 |
| | Ext Hold | 41.7/57.9 | 98.5/1.5 | 33.4/19.4/ |
| | 180 min | | | 47.2 |
| | Ext Hold | 41.2/58.3 | 98.6/1.3 | 29.2/20.8/ |
| | 240 min | | | 50.0 |
| | Ext Hold | 44.8/54.7 | 98.4/1.6 | 33.4/20.1/ |
| | 300 min | | | 46.4 |
| | Ext Hold | 55.7/44.2 | 98.2/1.8 | 31.3/14.3/ |
| | 360 min | | | 54.4 |

Immediate and extensive precipitation of protein was observed upon dilution of the thawed sample with SEC Mobile Phase A buffer (which is 0.1M sodium phosphate, 0.1M sodium sulfate, pH 6.8) in preparation for SEC analysis and remained steady in proportion to monomer throughout the subsequent five hours. Because this precipitation rendered the sample as a whole unsuitable for SEC, it was spun down and the supernatant (i.e. aggregated protein) was analyzed instead. This supernatant consisted mostly of HMW species. Immediate and stable shift toward basic species was also observed in the extended hold VI pool (Table 2).

Data from this hold experiment demonstrated that product aggregate reached unacceptably high levels, and gets worse over time, when the hold pH is less than 3.6. Notably, it was surprising that a hold pH of 3.5 yielded an unacceptably high level of aggregates as this pH is within a range typically used for viral inactivation of protein A eluate, suggesting that KPL-404 is particularly susceptible to aggreagation at and below this pH and that it was necessary to define a new pH range that does not endanger product quality.

### Run 2

Another run was executed with an experimental design as described below:
1) Experimental VI pH of 3.5. Control arm pH of 3.6. pH 3.6 is the upper end of the Process 2 range.
2) The control would consist of fully half of the ProA eluate instead of a small sample of it, thereby ensuring the volume would be large enough that samples of it could be neutralized appropriately prior to analysis.
3) QC planned to allow the samples to thaw and remain undisturbed for some time prior to dilution and testing, as this might prevent excessive aggregation.

The ProA eluate was 0.2 μm filtered and then adjusted to the target pH of 3.5 with 0.5M acetic acid. A control and initial ProA (pH 4.15) eluate samples were also taken. The control sample was adjusted to pH 3.6. All VI pools were held in PETG containers, titration was by pipette under constant mixing, and the holds were static at ambient temperatures. Every hour after the start of the hold, a 10 mL sample was taken from the experimental arm at pH 3.5, neutralized, and filtered. The control arm was sampled concurrently. Parameters for the VI operation are listed in Table 3.

TABLE 3
Operating Parameters, Run 2
Protein A Chromatography

| | | |
|---|---|---|
| Load Protein | 2,892 | mg |
| Eluate Protein | 2,801 | mg |
| Load Challenge (15-45 g/L) | 39.0 | g/L |

| | |
|---|---|
| Yield | 97% |

Viral Inactivation/Neutralization

| Experimental | | Control |
|---|---|---|
| VI pH | 3.50 | 3.60 |

| | |
|---|---|
| Sample Points | 60, 120, 180, 240, 300 min |

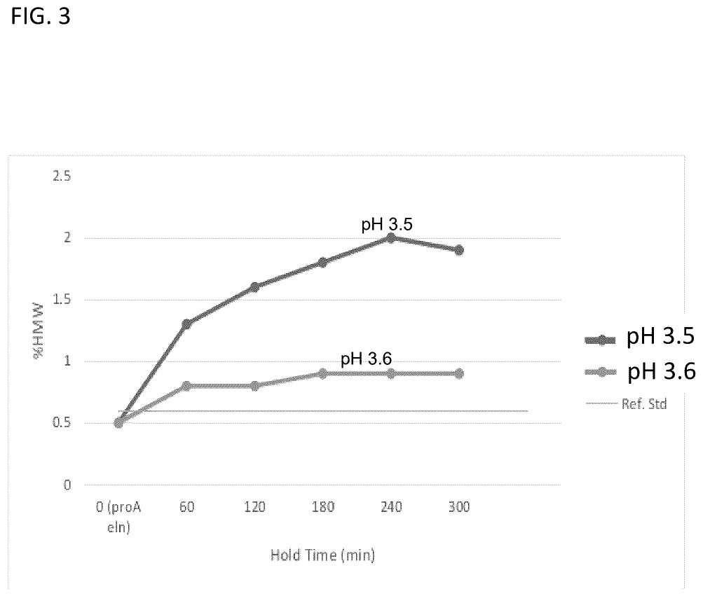

Run 2 data is presented in Tables 4-6. The percentage of high molecular weight (HMW) aggregates is shown in FIG. 3. Aggregation of KPL-404 was evident from the SEC data. It began promptly after acid addition, within 60 minutes, and there were indications it might begin the process of plateauing around the 240 minute mark.

IcIEF and NR-CESDS assays were also performed on the control and experimental arms to determine fragmentation or shift in charge distribution ou to 300 minutes. The results are shown in Tables 4-6.

TABLE 4
SEC Results

| | | | Peak ID/Average % Peak Area | | |
|---|---|---|---|---|---|
| # | | Sample Name | % HMW | % Monomer | % LMW |
| 1 | | Reference Standard | 0.6 | 99.4 | ND |
| 2 | pH 3.50 | KPL-404, 60 min | 1.3 | 98.7 | ND |
| 3 | | KPL-404, 120 min | 1.6 | 98.4 | ND |
| 4 | | KPL-404, 180 min | 1.8 | 98.2 | ND |
| 5 | | KPL-404, 240 min | 2.0 | 98.0 | ND |
| 6 | | KPL-404, 300 min | 1.9 | 98.1 | ND |
| 7 | pH 3.60 | KPL-404, 60 min | 0.8 | 99.2 | ND |
| 8 | | KPL-404, 120 min | 0.8 | 99.2 | ND |
| 9 | | KPL-404, 180 min | 0.9 | 99.1 | ND |
| 10 | | KPL-404, 240 min | 0.9 | 99.1 | ND |
| 11 | | KPL-404, 300 min | 0.9 | 99.1 | ND |
| 12 | | KPL-404 | 0.5 | 99.5 | ND |
| | | ProA Eluate | | | |

TABLE 5
icIEF Results

| | | Main | Total peak area % breakdown | | |
|---|---|---|---|---|---|
| # | Sample Name | Peak pI | % Acidic | % Main | % Basic |
| 1 | Reference | 8.1 | 34.3 | 50.0 | 15.7 |
| | Standard | | | | |
| 2 | KPL-404, 300 min | 8.1 | 27.9 | 47.2 | 24.9 |
| | (pH 3.50) | | | | |
| 3 | KPL-404, 300 min | 8.1 | 29.3 | 46.2 | 24.5 |
| | (pH 3.60) | | | | |
| 4 | KPL-404 ProA Eluate | 8.1 | 29.5 | 46.4 | 24.2 |

TABLE 6
NR-CE-SDS Results

| # | Sample Name | Total Impurities | IgG |
|---|---|---|---|
| 1 | Reference Standard | 1.0 | 99.0 |
| 2 | KPL-404, 300 min (pH 3.50) | 1.1 | 98.9 |
| 3 | KPL-404, 300 min (pH 3.60) | 1.1 | 98.9 |
| 4 | KPL-404 ProA Eluate | 1.1 | 98.9 |

In summary, this work within the broader panel of KPL-404 robustness studies comprised extended viral inactivation hold experiments, and evaluated the lower edge of the pH range (e.g., pH 3.3, 3.4, 3.5 and 3.6) to understand the risk to product quality. Despite that pH≤3.6 is typically the industry standard for the viral inactivation process, an unacceptable level of aggregation was observed for KPL-404 within this pH range. Accordingly, a new range for pH and/or hold time for viral inactivation has been developed to ensure that KPL-404 can be on VI hold for an extended period of time without compromising product quality.

### Example 2: Evaluation of a Revised pH Range for Virus Inactivation in KPL-404 Purification Process

As demonstrated in Example 1, the industry standard pH range for viral inactivation process was not acceptable for KPL-404 purification as a significant level of aggregates was observed in this range. In order to reduce protein aggregation during the viral inactivation step while minimizing the risk of reduced viral clearance capability, a revised pH range, i.e., pH 3.6-3.8, was proposed. Specifically, a pH of 3.85, at the upper end of the proposed VI pH range, was selected and evaluated in this Example. Table 7, shows SEC results of KPL-404 subjected to a VI hold at pH 3.8, demonstrating that a VI hold at pH 3.8 does not compromise KPL-404 product quality.

TABLE 7
SEC Results

| Sample | VI pH | % Monomer | % HMW | % LMW |
|---|---|---|---|---|
| KPL-404 | 3.8 | 99.4 | 0.6 | ND |
| KPL-404 | 3.8 | 99.5 | 0.5 | ND |

KPL-404 was produced from a stable CHO cell line. To assure the safety of CHO-derived products, it is necessary to evaluate their purification processes by spiking with model viruses to show removal or inactivation of several logs of viral infectivity. These studies may provide some assurance that adventitious agents, including viral contaminants that may be introduced by starting material (or materials employed during manufacturing), are not present in the purified product. In this Example, KPL-404 purification process was evaluated for its ability to inactivate Xenotropic murine leukemia virus (X-MuLV).

The following table summarizes the characteristics of this virus:

| Virus | Virus Family | Envelope | Geno | Approx. Size | Shape |
|---|---|---|---|---|---|
| X-MuLV | Retroviridae | Yes | RNA | 80-130 | Spherical |

Xenotropic murine leukemia virus (pNFS Th1 strain) is an 80-130 nm, enveloped, RNA-containing retrovirus. X-MuLV serves as a model for the retroviral particles that are frequently found in CHO cell lines. The virus was initially characterized by identification of the species host range [testing positive for growth on Mv1Lu cells (mink lung) and negative for growth on NIH3T3 cells (murine)]. The X-MuLV stock solution used in the current study tested positive for identity for X-MuLV and is free of potential bovine (BAV, BPV, BRSV, BTV, BVDV, IBR, and Reo-3), and porcine (PAV, PPV, and TGE) viral contaminants. The strength (titer) of the X-MuLV Ultra 2 stock virus (lot 03Jun20) used in this study was determined by a plaque assay utilizing PG4 indicator cells. The approximate titers of the X-MuLV stocks are typically between 5×10⁶ and 3×10⁷ PFU/ml.

For viral inactivation study, all times were ±1 minute. All temperatures were monitored and recorded. All samples were handled aseptically to avoid the introduction of contamination. All stock virus solutions were sonicated and filtered prior to spike.

### Low pH Treatment Samples

A portion of the Low pH Starting Material was adjusted to pH 3.80-3.90 (targeting 3.85), using Low pH Buffer, 0.5M Acetic Acid. The material was spiked to target 7.0-8.0 log10 total PFU with the appropriate stock virus solution (Ultra 2 X-MuLV) and re-adjusted to pH 3.80-3.90 (targeting 3.85), using Low pH Buffer, 0.5M Acetic Acid (if necessary). The spiked material was incubated for 120 minutes at 14.0±1.0° C. At 5 minutes, 15 minutes, 30 minutes, 60 minutes, 90 minutes, and 120 minutes following the initiation of incubation, a sample was removed, adjusted to pH 6.5-7.5 using Low pH Buffer, 0.5M Tris Base and filtered (0.45 μm). A portion of each sample was tested immediately by infectivity. The remaining material were divided into multiple aliquots, snap frozen and stored as back-ups at or below −60° C. Extra volume testing was performed for the “Low pH−T_{60 minutes}” sample to increase the sensitivity of the assay.

### Neutral Control Samples

An additional sample of the Low pH Starting Material was pH adjusted to pH 6.5-7.5 using Low pH Buffer, 0.5M Tris Base, spiked with the same % v/v ratio of the stock virus solution used to spike the treatment sample with 7.0-8.0 log10 total PFU and confirmed to be pH 6.5-7.5. An aliquot of the spiked Low pH Starting Material was immediately removed, confirmed to be pH 6.5-7.5 and filtered (0.45 μm). A portion of the sample was tested immediately by infectivity. The remaining material was divided into multiple aliquots, snap frozen and stored as back-ups at or below −60° C. This served as the “Low PH−T₀” sample.

The remaining spiked material was incubated for 120 minutes at 14.0±1.0° C. Following incubation, a sample was removed, confirmed to be pH 6.5-7.5 and filtered (0.45 μm). A portion of the sample was tested immediately by infectivity. The remaining material was divided into multiple aliquots, snap frozen and stored as back-ups at or below −60° C. This served as the “Low pH-Processing Control” sample.

The process was performed in duplicate. The following samples were generated and tested for the presence of X-MuLV by infectivity:

| | X-MuLV | X-MuLV |
|---|---|---|
| | Infectivity | Infectivity |
| | Samples - | Samples - |
| | Run 1 | Run 2 |
| 1 | Low pH - T₀ | Low pH - T₀ |
| 2 | Low pH - T_{5 minutes} | Low pH - T_{5 minutes} |
| 3 | Low pH - T_{30 minutes} | Low pH - T_{30 minutes} |
| 4 | Low pH - T_{60 minutes} | Low pH - T_{60 minutes} |
| 5 | Low pH - T_{90 minutes} | Low pH - T_{90 minutes} |
| 6 | Low pH - T_{120 minutes} | Low pH - T_{120 minutes} |
| 5 | Low pH - Processing Control | Low pH - Processing Control |

### Controls for Viral Removal Studies

### Stock Virus Controls

An aliquot of each sonicated and filtered stock virus solution that was used for spiking was tested within the appropriate plaque assay. This determined the starting titer of the stock virus solution used for spiking.

### Quantification of Infectious Virus via Plaque Assay

Upon initiation of testing, a portion of each test and control sample was diluted in serum-free medium to the end point (10⁰, 10⁻¹, 10⁻², 10⁻³, 10⁴, 10⁻⁵, 10⁶, 10⁻⁷, 10⁻⁸ and 10⁻⁹ as appropriate). Each appropriate dilution was assayed by the standard virus titration procedure, as described below for each virus.

X-MuLV: Each appropriate dilution was assayed in multiple wells and/or dishes for infectious viral particles by the X-MuLV plaque assay using PG4 indicator cells.

The following controls were tested within each plaque assay: for each virus, an aliquot of the sonicated and filtered stock virus solution, which served as an assay control, was used to generate the validity criteria for each assay. Serum-free medium was served as a negative control for each assay.

### Validity

The study was considered valid since the “Stock Virus Control” defined above displayed virus titers of greater than 1×10⁶ infectious units per ml. In addition, the negative controls were free of virus (i.e. no viral plaques observed).

### Process Results

Table 8 summarizes virus log₁₀ reduction values along with 95% confidence limits for samples that were subjected to a VI hold of 60 minutes at a pH of 3.85. Viral titers observed for samples that were subjected to a VI hold of 60 minutes at a pH of 3.6 are shown in Table 9, suggesting that the worst case viral clearance condition (i.e., pH 3.85) for the revised pH range (pH 3.6-3.8) resulted in an acceptable viral clearance capability. Viral titers were determined by multiplying the “Mean PFU” by the “Dilution” and dividing by the volume plated.

TABLE 8
Log₁₀ Reduction Summary pH 3.85

| Virus | Sample Designation | Log10 Reduction |
|---|---|---|
| X-MuLV | Low pH (3.85)- T_{60 minutes} - Run 1 | 3.45 ± 0.25 |
| | Low pH (3.85) - T_{60 minutes} - Run 2 | 5.85 ± 0.29 |

TABLE 9
Log₁₀ Reduction Summary pH 3.65

| Virus | Sample Designation | Log10 Reduction |
|---|---|---|
| X-MuLV | Low pH (3.65)- T_{60 minutes} - Run 1 | >5.90 ± 0.09* |
| | Low pH (3.65)- T_{60 minutes} - Run 2 | >5.87 ± 0.03* |

1. A method of producing a virus-inactivated antibody preparation comprising an anti-CD40 antibody, or antigen-binding portion thereof, having a reduced level of high molecular weight aggregates, the method comprising incubating a sample comprising the anti-CD40 antibody, or antigen-binding portion thereof, at a pH of about 3.6-3.9 during a viral inactivation step, thereby producing the virus-inactivated antibody preparation having a reduced level of high molecular weight aggregates.

2. A method of minimizing formation of high molecular weight aggregates in an antibody preparation comprising an anti-CD40 antibody, or antigen-binding portion thereof, during a viral inactivation step, the method comprising incubating a sample comprising the anti-CD40 antibody, or antigen-binding portion thereof, at a pH of about 3.6-3.9 during the viral inactivation step, thereby minimizing formation of high molecular weight aggregates in the virus-inactivated antibody preparation.

3. A method of reducing formation of high molecular weight aggregates in a antibody preparation comprising an anti-CD40 antibody, or antigen-binding portion thereof, during a viral inactivation step, the method comprising incubating a sample comprising the anti-CD40 antibody, or antigen-binding portion thereof, at a pH of about 3.6-3.9 during the viral inactivation step, thereby reducing the formation of high molecular weight aggregates in the virus-inactivated antibody preparation.

4. A method of maximizing the level of antibody monomers in a virus-inactivated antibody preparation comprising an anti-CD40 antibody, or antigen-binding portion thereof, the method comprising incubating a sample comprising the anti-CD40 antibody, or antigen-binding portion thereof, at a pH of about 3.6-3.9 during a viral inactivation step, thereby maximizing antibody monomers in the virus-inactivated antibody preparation.

5. A method of stabilizing a virus-inactivated antibody preparation comprising an anti-CD40 antibody, or antigen-binding portion thereof, the method comprising incubating a sample comprising the anti-CD40 antibody, or antigen-binding portion thereof, at a pH of about 3.6-3.9 during a viral inactivation step, thereby stabilizing the virus-inactivated antibody preparation.

6. A method of reducing the number and/or activity of viral particles in an antibody preparation comprising an anti-CD40 antibody, or antigen-binding portion thereof, the method comprising incubating a sample comprising the anti-CD40 antibody, or antigen-binding portion thereof, at a pH of about 3.6-3.9 during a viral inactivation step, thereby reducing the level the number and/or activity of viral particles in the antibody preparation.

7. The method of any one of claims 1-6, wherein the viral inactivation step comprises a virus inactivation period, and optionally a static holding period.

8. The method of any one of claims 1-7, wherein the anti-CD40 antibody, or antigen binding portion thereof, comprises a heavy chain variable region comprising a CDR1 having an amino acid sequence of SEQ ID NO:1, a CDR2 having an amino acid sequence of SEQ ID NO:2, and a CDR3 having an amino acid sequence of SEQ ID NO:3.

9. The method of any one of claims 1-8, wherein the anti-CD40 antibody, or antigen binding portion thereof, comprises a light chain variable region comprising a CDR1 having an amino acid sequence of SEQ ID NO:4, a CDR2 having an amino acid sequence of SEQ ID NO:5, and a CDR3 having an amino acid sequence of SEQ ID NO:6.

10. The method of any one of claims 1-9, wherein the anti-CD40 antibody, or antigen binding portion thereof, comprises a heavy chain variable region comprising an amino acid sequence of SEQ ID NO:7, and a light chain variable region comprising an amino acid sequence of SEQ ID NO:8.

11. The method of any one of claims 1-10, wherein the anti-CD40 antibody, or antigen binding portion thereof, comprises a heavy chain comprising an amino acid sequence of SEQ ID NO:9, and a light chain comprising an amino acid sequence of SEQ ID NO:10.

12. The method of any one of claims 1-11, wherein the anti-CD40 antibody or antigen-binding portion thereof is KPL-404.

13. The method of any one of claims 1-12, wherein the sample is incubated during the viral inactivation step at a pH of about 3.6-3.9, about 3.6-3.8, about 3.7-3.9, about 3.6-3.7, about 3.7-3.8, or about 3.8-3.9.

14. The method of any one of claims 1-13, wherein the sample is incubated during the viral inactivation step at a pH of about 3.6, about 3.7, about 3.8, or about 3.9.

15. The method of any one of claims 1-14, wherein the sample is incubated during the viral inactivation step for about 15-360 minutes.

16. The method of any one of claims 1-15, wherein the sample is incubated during the viral inactivation step for about 15-50 minutes, about 20-40 minutes, about 20-60 minutes, about 30-70 minutes, about 30-60 minutes, about 30-120 minutes, about 60-120 minutes, about 40-80 minutes, about 50-70 minutes, about 50-90 minutes, about 60-100 minutes, about 70-110 minutes, about 80-100 minutes, about 80-120 minutes, about 90-130 minutes, about 100-140 minutes, about 110-130 minutes, about 110-150 minutes, about 120-160 minutes, about 130-170 minutes, about 140-180 minutes, about 150-190 minutes, about 160-200 minutes, about 170-190 minutes, about 170-210 minutes, about 180-220 minutes, about 200-240 minutes, about 220-260 minutes, about 230-250 minutes, about 240-280 minutes, about 280-320 minutes, about 290-310 minutes, or about 320-360 minutes.

17. The method of any one of claims 1-16, wherein the sample is incubated during the viral inactivation step at a temperature of about 13° C.-37° C., about 15° C.-37° C., about 15° C.-30° C., about 13° C.-25° C., or about 15° C.-25° C.

18. The method of any one of claims 1-17, wherein the sample is incubated during the viral inactivation step at a pH of about 3.6-3.8 for about 30-120 minutes.

19. The method of any one of claims 1-18, wherein the sample is incubated during the viral inactivation step at a pH of about 3.7-3.8 at a temperature of about 13° C.-25° C. for about 30-120 minutes.

20. The method of any one of claims 1-19, wherein the sample is incubated during the viral inactivation step at a pH of about 3.7 for about 60-120 minutes.

21. The method of any one of claims 1-20, wherein the sample is incubated during the viral inactivation step at a pH of about 3.7 at a temperature of about 25° C. for about 120 minutes.

22. The method of any one of claims 1-21, wherein the sample is subjected to a Protein A chromatography column prior to the viral inactivation step.

23. The method of any one of claims 1-22, wherein the virus-inactivated antibody preparation comprises less than 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% high molecular weight aggregates.

24. The method of any one of claims 1-23, wherein the virus-inactivated antibody preparation comprises less than 2% high molecular weight aggregates.

25. The method of any one of claims 1-24, wherein the level of high molecular weight aggregates is determined by size exclusion chromatography.

26. A composition comprising an anti-CD40 antibody, or antigen-binding portion thereof, wherein the composition comprises a virus-inactivated eluate collected from an affinity chromatography column, wherein the eluate comprises less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% high molecular weight aggregates.

27. The composition of claim 26, wherein the virus-inactivated eluate comprises less than 2% high molecular weight aggregates.

28. The composition of claim 26 or 27, wherein the affinity chromatography column comprises a Protein A chromatography column.

29. The composition of any one of claims 26-28, wherein the virus-inactivated eluate has been subjected to a viral inactivation step at a pH of about 3.6-3.9.

30. The composition of any one of claims 26-29, wherein the anti-CD40 antibody, or antigen binding portion thereof, comprises a heavy chain variable region comprising a CDR1 having an amino acid sequence of SEQ ID NO:1, a CDR2 having an amino acid sequence of SEQ ID NO:2, and a CDR3 having an amino acid sequence of SEQ ID NO:3.

31. The composition of any one of claims 26-30, wherein the anti-CD40 antibody, or antigen binding portion thereof, comprises a light chain variable region comprising a CDR 1 having an amino acid sequence of SEQ ID NO:4, a CDR2 having an amino acid sequence of SEQ ID NO:5, and a CDR3 having an amino acid sequence of SEQ ID NO:6.

32. The composition of any one of claims 26-31, wherein the anti-CD40 antibody, or antigen binding portion thereof, comprises a heavy chain variable region comprising an amino acid sequence of SEQ ID NO:7, and a light chain variable region comprising an amino acid sequence of SEQ ID NO:8.

33. The composition of any one of claims 26-32, wherein the anti-CD40 antibody, or antigen binding portion thereof, comprises a heavy chain comprising an amino acid sequence of SEQ ID NO:9, and a light chain comprising an amino acid sequence of SEQ ID NO:10.

34. The composition of any one of claims 26-33, wherein the anti-CD40 antibody or antigen-binding portion thereof is KPL-404.

35. A method of producing a pharmaceutical composition comprising an anti-CD40 antibody and a pharmaceutically acceptable carrier, the method comprising incubating a sample comprising the anti-CD40 antibody, or antigen-binding portion thereof, at a pH of about 3.6-3.9 during a viral inactivation step, thereby producing the pharmaceutical composition comprising the anti-CD40 antibody and the pharmaceutically acceptable carrier.

36. The method of any one of claims 1-34, wherein the antibody preparation comprises charged species of the anti-CD40 antibody, wherein the charged species comprises at least about 40%, 41%, 42%, 43%, 44%, 45%, 46%, 47%, 48%, 49%, or 50% main species.

37. The method of claim 35, wherein the pharmaceutical composition comprises charged species of the anti-CD40 antibody, wherein the charged species comprises at least about 40%, 41%, 42%, 43%, 44%, 45%, 46%, 47%, 48%, 49%, or 50% main species.


SEQUENCE LISTING (12 sequences)

SEQ ID NO: 1 - protein, 9 aa, synthetic construct
  REGION 1..9: note = Synthetic: Heavy chain, CDR1
YTFTNYWMH

SEQ ID NO: 2 - protein, 17 aa, synthetic construct
  REGION 1..17: note = Synthetic: Heavy chain, CDR2
YINPSNDYTKYNQKFKD

SEQ ID NO: 3 - protein, 5 aa, synthetic construct
  REGION 1..5: note = Synthetic: Heavy chain, CDR3
QGFPY

SEQ ID NO: 4 - protein, 10 aa, synthetic construct
  REGION 1..10: note = Synthetic: Light chain, CDR1
SASSSVSYMH

SEQ ID NO: 5 - protein, 7 aa, synthetic construct
  REGION 1..7: note = Synthetic: Light chain, CDR2
DTSKLAS

SEQ ID NO: 6 - protein, 9 aa, synthetic construct
  REGION 1..9: note = Synthetic: Light chain, CDR3
HQLSSDPFT

SEQ ID NO: 7 - protein, 114 aa, synthetic construct
  REGION 1..114: note = Synthetic: Heavy chain, variable region
QVQLVQSGAEVKKPGASVKVSCKASGYTFTNYWMHWVRQAPGQRLEWIGYINPSNDYTKYNQKFKDRATLTADKSANTAYMELSSLRSEDTAVYYCARQGFPYWGQGTLVTVSS

SEQ ID NO: 8 - protein, 106 aa, synthetic construct
  REGION 1..106: note = Synthetic: Light chain, variable region
EIVLTQSPATLSLSPGERATLSCSASSSVSYMHWYQQKPGQAPRRWIYDTSKLASGVPARFSGSGSGTDYTLTISSLEPEDFAVYYCHQLSSDPFTFGGGTKVEIK

SEQ ID NO: 9 - protein, 441 aa, synthetic construct
  REGION 1..441: note = Synthetic: Heavy chain (with IgG4 constant domain)
QVQLVQSGAEVKKPGASVKVSCKASGYTFTNYWMHWVRQAPGQRLEWIGYINPSNDYTKYNQKFKDRATLTADKSANTAYMELSSLRSEDTAVYYCARQGFPYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 10 - protein, 213 aa, synthetic construct
  REGION 1..213: note = Synthetic: Light chain (with kappa constant domain)
EIVLTQSPATLSLSPGERATLSCSASSSVSYMHWYQQKPGQAPRRWIYDTSKLASGVPARFSGSGSGTDYTLTISSLEPEDFAVYYCHQLSSDPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 277 aa, Homo sapiens
  REGION 1..277: note = NP_001241.1 tumor necrosis factor receptor superfamily member 5 isoform 1 precursor
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 12 - protein, 203 aa, Homo sapiens
  REGION 1..203: note = NP_690593.1 tumor necrosis factor receptor superfamily member 5 isoform 2 precursor
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTRSPGSAESPGGDPHHLRDPVCHPLGAGLYQKGGQEANQ